SEQUENCE LISTING (15 sequences)

SEQ ID NO: 1 - protein, 311 aa, synthetic construct
MSKVVFSSLKEWKQRALRMHFQGLTSSEIATELGISDRTIRDNIQKLAHRVDKSVVLKPRKPTIFVIGDTQVKQGISLDYIHWIANYIKLKQPDIIVQIGDHYDMASLSTYDKGQLSAEGRRFVLDIEAGDEALGIIEDYIRSVKGYNPRKVVVLGNHEDRIDRFVKTHPEFEGLIGTDKLAFHDYGWEVIPFLKPHNICGIHFVHYAVNVNTGKPLGGNLDLRLKTVGESFVMGHQQMYAYAERQLPMTGRKQFAAVVGACYVHDEPYKGWQGNHHFRGCLMLYGCADGYAMQKKVELEHMREIYEGEHA

SEQ ID NO: 2 - protein, 149 aa, synthetic construct
MDCIDHGMAGDKDGYGSTCAHEYKGERNKLKLHRLAYCRANNLHIKDIDGKVVMHTCDNPRCINPEHLKLGSVADNNRDRVSKGRYCGSQSIRAKLTDADIAFIRANYKPRSKVWNTHTLAKKFGVVHQTISVITRNIRYKEQVGVTRI

SEQ ID NO: 3 - protein, 645 aa, synthetic construct
MIDATFRERFKRLRAHVAQHNDRPELIPKEDREMFALMFAGLFLSFRDFAELGMAYLGFKMSEIQDDIADYMQHGYKYRMVQAQRGQAKSTLAALYCIWRLIQRPKDRCLIVSAGGDQADSIALIITRIINQWDILCWMRPDTTRGDRDSAKNYDIHCDLKGIDKSASVSSVGISAQLAGKRADFLLADDIEVMRNSMTQTEREKLALQTREFSAICIHGDIMYLGTPQTKDSIYRELPRRGFSVRVWTGRYPTNEELERYGSGTEIAPMIMKRLLENPELQTGGGIEGNRGQPTDPNHIGEETLQSKELDYGPEGFALQYMLDTTLSDEMRTKIKLSDIPVVGTGTDSAPEVVQYKCDPTTAYKELTPAMTAFRMYWGIGSDKSVPFEHKVMIIDPAGSGGDEIAFATGAATNSYIYLLSVGGFKGGTKEENLNKVIMKMVTSGIKDLDIERNMGHGTVTQLVVAQIEKLRLKASKGSQDEDFLELLQSYGVTHSELNSALSGVAVNDYFVTTQKERRIIDTISPVTRRHKLVVTSSAIQEDWEYCLQHPMEKRNQYSCFYQLGNITYDRGSLVHDDRADCVQRLVERLSPFLAKDDEAGAVKRREEEIAEWRRNPMGYTHGKFANTGMRRGSGTTRKFGGRRK

SEQ ID NO: 4 - protein, 185 aa, synthetic construct
MILTKDGFSIIRNELFGGKLDQTQVDAINFIVEKATESGLTYPEAAYLLATIYHETGLPSGYRTMQPIKEAGSDSYLRSKKYYPYIGYGYVQLTWKENYGRIGKLIGIDLVKNPEKALEPLIAIQIAIKGMLNGWFTGVGFRRKRPVSKYNKQQYVAARNIINGKDKAELIAKYAIIFERALRSL

SEQ ID NO: 5 - protein, 518 aa, synthetic construct
MKSKGNDFTKTIRALYDEYTDDSLKTRLEMYALWTLPSVFPTGEITVDNGNAEIEHDYQSVGAYLVNRLASRLASTLFPVSTSFFRIEPSQELKDLVDKRGTSTLIDLENKACRRLFFNASYAQIVQALRLLIITGEVLLLRRDNRLRVFSLKNYALLRNNVGEVLEIITREPKRYRELDAETQELLQDRNEDETLDLYTRIRKRNINGVISWKITQEIDGVRLPNYEIYRDKLCPYIPVTWSYMNGDAYGRGYVEEYAGDFAKLSELSQGLTEYQIESLIIRHVYNAQGGFDVESAVNSRNGDWISGNVNAVQNYESGSFQKMNEVRLGLEAIMQRLNVAFMYTGNMREGDRVTAYEIARNADEAEQVLGGVYSQLSQNMHLPLAYLLLYEVRKDFIQAIDRQEIELNILTGLQALSRSSENQALLVAANEIATVAQVFSQVSKRFNLDAIVDKILLSNGIDISEITYSEEEMRAKAMEEQRAAEAQRQQVIQQAGAQLGGNQLENTQAAQLAAGIQ

SEQ ID NO: 6 - protein, 241 aa, synthetic construct
MSLQSRKDLQSFLKTKGFYFGNIDGIIGRGSKNAILACFQNKKAVAITTSELLTFAKELGDTNTVRIRAVAKVESAGAGWFKDGRPKILYERHKFWKYNDDISAPKSTFFNFPEGGNYTIDADNNGINDSYDKLLKACEYDPRGAFQAVSMGAFQVMGFYYKEMGFKEPWEMLFACTHSEKAQYDLLVKYIKMNNLQGAFLKLSSRPDDCRPLARGYNGKNYVKFNYHGKLASAVLYFGGK

SEQ ID NO: 7 - protein, 97 aa, synthetic construct
MSKTILFPVGDRVLVKRKEKEDKSAGGIILSKEAQETEQQGEIILVGELIQQKNILKAGVMAHFGQYAGTNIKHEGEVFVLLKESDILAVTVGDDRV

SEQ ID NO: 8 - protein, 354 aa, synthetic construct
MAIRNPLVTINNQNYIQNIGDTHLGRVFKNNVALDRRGEWEKQQMVSLDEELFKTMPIGVESQNYIRVQAGDLFDKPIISNEVLMDTVKILKDYEDCKEEPLYIISGNHDDSKNESDVTSWDLLAKFFEGSKLIHFVKTWKAHVFPDGSTVLLVGWNIHESVATAYLQAYECGVNPAVVVCHLDRISYGNEDNVIPYDFLKQHHVDMVISGHEHKPYFFEEGHMKVIGTGSLLPYSHAEDPEGYFYQTYKGWDAFHEVLCNKGSDYFKYKHVRVYLEDDHMEDFAELNLSCYSLQVHKMERLQKDQELEGETEVIIESYNAKNIWQAAAQEVALPEDQSQMLWAEIEMEGIENE

SEQ ID NO: 9 - protein, 312 aa, synthetic construct
MKINVKAVMDYLFFNADTIQTKVKLLEATKCTETGRKLWTFELFFPRCILAEQNTHRKFSRNTGSSRAIPVMKMLKTLMLIPFIPLFWGLNKSGMQSAETMSDTKATLAAMLWKAHFWFTYMIVYLLSKLGLHKQWTNRLLEPHSYIRQVVTTDDWENYFNLRIHDDAQPEIILLALMIREQVELVERNNSFRMVSNKKALYTAENWHLPYITDKERKKYRAFPFYLAKISAARCARTSYLTQEGAVPNLEREIGTFKKLAESTPIHASPLEHQAYPAVNGETQSRNFQGFIQYRAYYEEKEFGKPIEIPNF

SEQ ID NO: 10 - protein, 650 aa, synthetic construct
MLLEHILDLSRDDNLTNFGKATLEDRYLKPNETSPQHAFLRASTAFASNEAHATRMYGYASKQWIGYASPILSNAPVRTKFAQAWEDNFGRDCFLSVVGALPISCFLGYVGDSREEIGFHYYETMWLASNGGGAKFYWGDLRPVNSETSTGSKTGGLIPFFHVDDGLIVATHQGNNRRGVDGGSIRDDHPEIVEFVESRKMSGDPNKRSRNLFQTVNLSDDFMYAVLGDKSWDLIDHKGIVSKTVQARYLWELMLETPFETGCPFLHWIDTSNDKLPPTQKARGLRTNTVNICTEITLPTDKDRTAVCCLSSINMLKHDEWINDPQFIADVTEFLDNVLEYFIQNAIYTCTKDFDWEKLKDTLRSELKEDNLDEDRIDQLAHKIVERNIMGYKKAVYSAKQERAIGIGMMGLDSYFMANEIAYESQEALTITQKTFRHIKAQAHAASVALGTLRGEAPDMEGTGRRNSHLLAIAPTATNSTIAGGATPALEKRYENIYTQKTKSGTFTVVEPALVKVLDKYGINTPEVIKSIKDNNGSVQHLRELTNHERLYLRTAFEVDQVWVVEHAATAQEEVCQAISCNLFFMPDVPRFYVNNVHLHMWKRGLKSRYYVRTKAVKSGDAFADALIKRIQTIDYKKHEYFDACPSCEG

SEQ ID NO: 11 - protein, 316 aa, synthetic construct
MKAYHDLLQKILDEGEEVTNERTGVGTLSIFAEQIKFDLREGFPAVTTKKLAFKSMIAELLWFIRGSSNLYDLRALTHGEEHRFNDEKKTIWDDNYNVQGKELGYSNGYCGLVYGTQWRNFGPTGVNVEEDFGGTYYHDVPGVDQLAEMIKEAKANPGSRRLIVEAWNPQLIWEHKTLPGYESHYEIQKPILPPCHTGFQINISGEFIDMIWHQRSVDTFLGLPFNIASYATLLRIFAQILGKTPRYLTGQLGDVHIYKNHIEQVKEQLTREEYDLPILTINPNLKTLEDFEKAVVGDFTLIGYKHHEAIKAQMAA

SEQ ID NO: 12 - protein, 183 aa, synthetic construct
MKPTTFMQIAYLISQESKCVSWKVGAVIAKNGRIISTGYNGSPAGGVNCCDHAESEGWTTNQYNPIPPHTNRTLLKGECRNLHSAWAAINEIHAELNAILYAAKNGTSIDGATMYVTLSPCPDCAKAIANSGIKQLVYSETYDRNIEGWEKILEAAGIKVFQINKQSLKNIDWQNIVNYGGEQ

SEQ ID NO: 13 - protein, 196 aa, synthetic construct
MSNVLFLGDAHLGHGNVARFRTQFRDEQDHFEHVEKVYYGAVTRRDVCYFTGDAVFTLERAKQVAKWPGKKILVCGNHDTDNLSMKQLCEVFDDVKALVKYKEFWLSHAPMHPSELRGKFNIHGHVHFATVDDCRYVNTSLEATDFKPIALYQVRDIIKARIKFYENVYSSYKTHLVPLPKFDVHAFAHEIPVGLK

SEQ ID NO: 14 - protein, 147 aa, synthetic construct
MIKQYVNYDLLDAFEHNDFDAIVHGCNCFHTMGAGIAGAIAKRFPVAVEADKKTEYGDWSKLGDVSIATTVNGDIINAYTQYRPGKCPTHQLYENIRAAFTLINSKYAGKSLGIPKIGAGIAGGNWQEIAEIINDVTPDVKIYVLYI

SEQ ID NO: 15 - protein, 187 aa, synthetic construct
MDVKPFFDAARELAGGRLTQAQVDELNKVVNRLVPDDVDISDLGVDLIRQFEGLRTRAYQDSVGIWTIGYGTIRYPNGVAVKAGDVCTEEQAKSYMKHDLQKFVKAVNKLVTVPLKQTQFDALVSLVYNIGEGAFAGSTLLKKLNSKDYAGAAAQFLVWNKGRVKGKLEVIPGLTNRRKKEKAYFEQ

(19) United States
(12) Patent Application Publication
(10) Pub. No.: US 2026/0364130 A1
(43) Pub. Date: Nov. 19, 2026

(54) ACINETOBACTER BAUMANNII PHAGES

(71) Applicant: Helmholtz Zentrum München Deutsches Forschungszentrum für Gesundheit und Umwelt (GmbH), Neuherberg (DE)

(72) Inventors: Li Deng, Neuherberg (DE); Mohammadali Khan Mirzaei, Neuherberg (DE); Jinling Xue, Neuherberg (DE)

(21) Appl. No.: 19/132,581

(22) PCT Filed: Nov. 28, 2023
(86) PCT No.: PCT/EP2023/083257

§ 371 Date: May 23, 2025

(30) Foreign Application Priority Data
Nov. 28, 2022 (EP) 22209797.4

(51) Int. Cl. A61K 35/76; A61P 31/04; C12N 7/00

(52) U.S. Cl. CPC A61K 35/76; A61P 31/04; C12N 7/00; C12N 2795/00021; C12N 2795/00032; C12N 2795/00051

(57) ABSTRACT

The present invention relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 1 and further comprise a nucleic acid being part of its genome, which hybridizes under stringent conditions to a signature sequence or its complementary sequence hereto comprising a nucleic acid sequence encoding an amino acid sequence as depicted in SEQ ID NO: 11, wherein the phages comprise a nucleic acid being part of its genome, wherein the nucleic acid comprises a nucleic acid sequence, which is at least 60% identical to the nucleic acid sequence encoding the amino acid sequence as depicted in SEQ ID NO: 1 and 11 and wherein the isolated phages are capable of infecting Acinetobacter baumannii. The present invention also relates to a pharmaceutical composition comprising said composition, and to said composition or said pharmaceutical composition for use in therapy as well as for use in a method of treating or preventing an infection of a subject with Acinetobacter baumannii. Additionally, the present invention relates to kits and to a method of preparing said composition comprising isolated phages and a composition comprising isolated phages obtainable or obtained by said method.

### CROSS-REFERENCE TO RELATED APPLICATIONS

The present application claims the benefit of priority of EP Patent Application No. 22 209 797.4 filed 28 Nov. 2022, the content of which is hereby incorporated by reference in its entirety for all purposes.

### FIELD OF THE INVENTION

The present invention relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 1, and further comprise a nucleic acid being part of its genome, which hybridizes under stringent conditions to a signature sequence or its complementary sequence hereto comprising a nucleic acid sequence encoding an amino acid sequence as depicted in SEQ ID NO: 11, wherein the phages comprise a nucleic acid being part of its genome, wherein the nucleic acid comprises a nucleic acid sequence, which is at least 60% identical to the nucleic acid sequence encoding the amino acid sequence as depicted in SEQ ID NO: 1 and 11 and wherein the isolated phages are capable of infecting Acinetobacter baumannii. The present invention also relates to a pharmaceutical composition comprising said composition, and to said composition or said pharmaceutical composition for use in therapy as well as for use in a method of treating or preventing an infection of a subject with Acinetobacter baumannii. Additionally, the present invention relates to kits and to a method of preparing said composition comprising isolated phages and a composition comprising isolated phages obtainable or obtained by said method.

### BACKGROUND OF THE INVENTION

The spread of multi-resistant bacteria is a global human health threat, according to the World Health Organization (WHO)(Chandler, 2019, Palgrave Communications 5, 1-13). One way to tackle this threat is the development of new therapeutics as the development of novel antibiotics is both slow and expensive (Altamirano and Barr, 2019, Clinical Microbiology Reviews 32). Members of the genus Acinetobacter are ubiquitous in the environment and the multiple-drug resistant species Acinetobacter baumannii is of significant clinical concern. Acinetobacter often causes infections in people who have weakened immune systems, such as hospitalized or very sick patients. Acinetobacter infections usually affect organ systems, which have a high content of fluid (e.g. peritoneal fluid, respiratory tract, urinary tract, cerebrospinal fluid etc.) leading to infections related to continuous ambulatory peritoneal dialysis and nosocomial pneumonia. Acinetobacter is often susceptible or resistant to many antibiotics, so that the threat level was escalated to urgent because of the emergence of easily spread resistance in Acinetobacter and the lack of current antibiotics, and antibiotics in development, to treat these infections.

Antimicrobial resistance (AMR) is a major threat to global health and global economies. Worldwide, infections by multiple-drug resistant bacteria are estimated to kill more than 700,000 people, including 25,000 cases in Europe and 23,000 cases in the US every year (Laximinarayan et al., The Lancet Infect Diseases (2013), 13: 1057-1098). Antimicrobial resistance also places a tremendous burden on healthcare systems and society, with an annual cost due to healthcare expenditures and productivity losses estimated by the European Commission at approximately €1.5 billion in the EU.

To tackle this challenge, bacteriophages (also called phages), viruses that infect bacteria, can be employed to fight AMR as a complement to antibiotic therapy (Salmond et al., Nat Rev Micro (2015), 13: 777-786). Studies revealed that phages are the most abundant biological entities on earth, with ~10³¹ phages in total and about 10²³ phage infections per second. Phages are highly specific in their choice of bacterial hosts, mediated by specific receptor proteins, thus will cause no collateral damage to the microbial companions that form our microbiome, which is increasingly appreciated as essential to our health (Shreiner et al., Curr Op Gastroenterol (2015), 31: 69-75). After over a billion years of co-evolutionary struggle with their bacterial hosts, phages have evolved highly diverse proteins that inhibit or ‘hijack’ bacterial metabolic processes to their own benefit.

In sum, a major concern associated with phage therapy is still the emergence of phage-insensitive mutants (BIMs) such as Acinetobacter baumannii.

There is thus an ongoing need in the art to provide an alternative or even improved phage therapy for infections associated with Acinetobacter baumannii. The technical problem underlying the present application is thus to comply with this need. The technical problem is solved by providing the embodiments reflected in the claims, described in the description and illustrated in the examples and figures that follow.

### SUMMARY OF THE INVENTION

The inventors successfully identified and characterized a composition comprising phages which have been isolated from samples inter alia by the proprietary high-throughput “viral tagging” as it is disclosed by WO2021/048257 incorporated by references herein and by classical phage isolation methods, and which phages comprised by the composition of the invention are against a multidrug resistant Acinetobacter baumannii (short. A. baumannii) bacteria so that such phages are capable of infecting A. baumannii. This results in replication of phage genome within said host cell and lysis of such bacteria. Most importantly, the major problem associated with phage therapy, namely the emergence of phage-insensitive mutants (BIMs) such as A. baumannii which are bacterial mutants of phage-sensitive bacteria, due to mutations affecting bacterial-surface molecules, thereby interfering with the phage attachment, and thus being phage resistant, could be solved by preventing the development of Acinetobacter resistance with the particular composition of the present invention comprising said isolated phages. Using the composition of the invention, the inventors were also able to mitigate biofilm formation in the A. baumannii strain, thus resulting in an efficient bacterial removal of the multiple-drug resistant A. baumannii and therefore minimizing the abovementioned problems with phage resistance and also AMR. The isolated A. baumannii phages comprised within the composition of the invention all hybridize with phage nucleic acid to (a) so called hook(s) which was/were designed by the inventors for the targeted A. baumannii phage isolation as explained in more detail below. Particularly, the isolated A. baumannii phages comprised within the composition of the invention preferably all hybridize with phage nucleic acid to two particular hooks as defined by the amino acid sequences of SEQ ID NOs: 1 and 11 (see FIG. 3D).

Accordingly, in a first aspect, the present invention comprises a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 1, and wherein the composition further comprises isolated phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 11, wherein the phages comprise a nucleic acid being part of its genome, wherein the nucleic acid comprises a nucleic acid sequence, which is at least 60%, at least 65%, at least 70%, at least 75%, at least 80%, at least 85%, at least 90%, at least 95%, at least 96%, at least 97%, at least 98%, at least 99%, at least 99.5%, or 100% identical to the nucleic acid sequence encoding the amino acid sequence as depicted in SEQ ID NO: 1 and 11 and wherein the isolated phages are capable of infecting A. baumannii.

Said signature sequence or its complementary sequence comprising a nucleic acid sequence encoding an amino acid sequence as depicted in any one selected from the group consisting of SEQ ID NOs: 1-15, the isolated phages hybridize under stringent conditions to, are considered as the so called “hook(s)”. In a first step of designing such hooks, the phages comprised in a sample as defined elsewhere herein were labeled by the viral tagging procedure as defined by WO2021/048257, before target bacteria (A. baumannii) were added into the samples comprising the labelled phages for phage isolation. After isolation of said A. baumannii phages by flow cytometry and sorting, A. baumannii bacteria were lysed to extract viral DNA as explained further in the Example section and the phage genome was then analyzed performing VT metagenomics sequencing and bioinformatics analysis (including protein clustering) as also described in the Examples. The genome of the isolated Acinetobacter phages, viral contigs generated by said viral tagging as defined by the Examples, and the Acinetobacter phage genomes from the NCBI RefSeq database were then used to create a gene catalogue, where the proteins of these genes were clustered into protein families and annotated. The most prevalent protein families within a viral cluster which was created by clustering said phage genome to genus level were used for designing said particular “hook(s)” the phages hybridize to and which hook(s) can then be applied for further targeted isolation of phages from an A. baumanii phage colony (e.g. by applying qPCR wherein the defined hooks can be/were used as primers after viral tagging of phages comprised by a sample as defined elsewhere herein and infecting target bacteria A. baumannii with such labeled phages within such sample as defined in more detail elsewhere herein). Such hooks—as can be seen below—may thus be used to fish out/isolate particular phages within an A. baumanii phage colony which hybridize to said specific hook(s). The hybridization of the A. baumannii phages to said defined hook(s) results in an isolation of divergent phages since each hook was designed for different phages, but which are all capable of infecting A. baumannii. For example the hook as defined by said signature sequence or its complementary sequence comprising a nucleic acid sequence encoding an amino acid sequence as depicted in SEQ ID NO: 1 refers to a protein family of a metallo-phosphoesterase which is one of the most abundant proteins within the A. baumannii phages which have been analysed by metagenomics sequencing. Meaning a targeted A. baumannii phage isolation using such particular hook allows to isolate the most divergent phages which are then comprised in said composition of the invention.

In a second aspect, the present invention further encompasses a pharmaceutical composition comprising said composition as defined elsewhere herein.

The invention additionally comprises in a third aspect said composition as defined elsewhere herein or said pharmaceutical composition for use in therapy. In a fourth aspect, the invention envisages said composition or said pharmaceutical composition as defined elsewhere herein for use in a method of treating or preventing an infection of a subject with A. baumannii.

In a fifth aspect, the present invention also comprises a kit comprising the composition as defined elsewhere herein or the pharmaceutical composition as defined elsewhere herein.

Accordingly, in a sixth aspect, the present invention also comprises a kit comprising a first nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 1, or a complementary sequence thereof, and further comprising a second nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 11, or a complementary sequence thereof. Preferably, the present invention comprises a kit comprising a first nucleic acid comprising a nucleic acid sequence, which is at least 90% identical to a nucleic acid sequence encoding an amino acid sequence as depicted in SEQ ID NO: 1 or a complementary sequence thereof, and further comprising a second nucleic acid comprising a nucleic acid sequence, which is at least 90% identical to a nucleic acid sequence encoding an amino acid sequence as depicted in SEQ ID NO: 11, or a complementary sequence thereof.

In a seventh aspect, the invention also encompasses a method of preparing a composition comprising isolated phages, the method comprising:
targeted isolating of phages comprised in a provided sample, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and further comprise a nucleic acid being part of its genome, which hybridizes under stringent conditions to a signature sequence or its complementary sequence hereto comprising a nucleic acid sequence encoding an amino acid sequence as depicted in SEQ ID NO: 11, and wherein the phages comprise a nucleic acid being part of its genome, wherein the nucleic acid comprises a nucleic acid sequence, which is at least 60%, at least 65%, at least 70%, at least 75%, at least 80%, at least 85%, at least 90%, at least 95%, at least 96%, at least 97%, at least 98%, at least 99%, at least 99.5%, or 100% identical to the nucleic acid sequence encoding the amino acid sequence as depicted in SEQ ID NO: 1 and 11.

Finally, the present invention envisages in an eighth aspect a composition comprising isolated phages obtainable or obtained by the method as defined above.

### BRIEF DESCRIPTION OF THE FIGURES

FIG. 1: A heatmap of the presence-absence matrix shows protein families (x-axis) in different viral clusters (y-axis).

FIG. 2: (A) Genome of vB_AbM_HMGU, vB_AbS_HMGU, and vB_AbA_HMGU; arrows indicate the predicted CDSs and the directions of arrows indicate the direction of transcription. (B) Transmission electron microscopy of vB_AbS_HMGU. (C) Infection kinetics of vB_AbS_HMGU such as adsorption rate, latency period, and burst size of vB_AbS_HMGU.

FIG. 3: The fluorescent staining of the bacterial biofilm under different treatment scenarios. Live cell (green, depicted as arrow) and dead cells (red, depicted with circles); (A) negative control; (B) phage vB_AbM_HMGU; (C) vB_AbA_HMGU; (D) combination of phages vB_AbM_HMGU and vB_AbA_HMGU.

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments described throughout the specification should not be construed to limit the present invention to only the explicitly described embodiments. This description should be understood to support and encompass embodiments which combine the explicitly described embodiments with any number of the disclosed and/or preferred elements. Furthermore, any permutations and combinations of all elements described herein should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. When used herein the term “comprising” can be substituted with the term “containing” or “including” or sometimes when used herein with the term “having”. When used herein “consisting of” excludes any element, step, or ingredient not specified.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless otherwise indicated, the term “at least” preceding a series of elements is to be understood to refer to every element in the series. The term “at least one” refers to one, two, three or more such as four, five, six, seven, eight, nine, ten and more. Those skilled in the art will recognize, or be able to ascertain using no more than routine experimentation, many equivalents to the specific embodiments of the invention described herein. Such equivalents are intended to be encompassed by the present invention.

The term “less than” or in turn “more than” or “below” does not include the concrete number.

The term “and/or” wherever used herein includes the meaning of “and”, “or” and “all or any other combination of the elements connected by said term”.

When used herein “consisting of” excludes any element, step, or ingredient not specified in the claim element. When used herein, “consisting essentially of” does not exclude materials or steps that do not materially affect the basic and novel characteristics of the claim.

The term “including” means “including but not limited to”. “Including” and “including but not limited to” are used interchangeably.

The term “about” means plus or minus 20%, preferably plus or minus 10%, more preferably plus or minus 5%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the examples, offered for illustrative purposes only. The examples are not intended to limit the scope of the present invention in any way.

### Composition

A “composition” comprising the phages which have been isolated as defined elsewhere herein, refers to any kind of composition which comprises these isolated phages, e.g. naturally-occurring/environmental samples, a nutritional composition, a cosmetic composition or the like. Said composition can be a liquid (preferably aqueous), a solid, a gel, a powder, a paste, an ointment, a capsule, a food product etc. Further comprised herein is a dried or frozen form of the composition as defined herein. Thus, said composition may be stored directly in liquid form for later use, stored in a frozen state and thawed prior to use, or prepared in dried form, such as a lyophilized, air-dried, or spray-dried form, for later reconstitution into a liquid form or other form prior to use. Thus, it is envisaged that a composition described herein may be stored by any method known to one of skill in the art. Non-limiting examples include cooling, freezing, lyophilizing, and spray drying the formulation, wherein storage by cooling is preferred.

The composition of the invention comprising said isolated phages may further comprise one or more ingredients, by the way of illustration and not limitation, such as an excipient, a preservative, an ingestible support, a flavour, a solubilizer, a wetting agent, a sweetener, a colorant, a coating agent, or an antioxidant. The excipients of the composition may refer to diluents such as, e.g. water, saline, glycerol, ethanol, bacteriostatic water for injection (BWFI), Ringer's solution, dextrose solution, or aqueous solutions of salts and/or buffers etc. Thus, the composition of the present invention may further comprise at least one excipient as defined herein, preferably a buffer (pH buffering agent) as excipient. Furthermore, substances necessary for formulation purposes may be comprised in said composition as acceptable excipients such as emulsifying agents, stabilizing agent, and/or surfactants known to a person skilled in the art.

The term “buffer” or “pH buffering agent” as used herein, includes those agents that maintain the pH in a desired range. A buffer is an aqueous solution consisting of a mixture of a weak acid and its conjugate base or a weak base and its conjugated acid. It has the property that the pH of the solution changes very little when a small amount of a strong acid or base is added. Buffer solutions are used as a means of keeping pH at a nearly constant value in a wide variety of chemical applications. A buffer when applied in the composition of the invention preferably stabilizes the isolated phages. Preferably, as a buffer being further comprised by the composition of the present invention PBS or sodium bicarbonate buffer is used.

As used herein, the term “isolated” refers to a substance and/or entity that has been (1) separated from at least some of the components with which it was associated when initially produced (whether in nature and/or in an experimental setting), and/or (2) designed, produced, prepared, and/or manufactured by the hand of man. In the context of the present invention, the phages which are isolated may refer to the substance and/or entity separated from samples such as sewage water samples by applying at least any one of the defined hooks which have been explicitly designed/prepared by the inventors as explained above and in the Example section in more detail. Such “targeted phage isolation” (“hook isolation”) refers to applying viral tagging as defined in the Example section and as it is disclosed by WO2021/048257, followed by phage isolation methods as defined elsewhere herein and qPCR, where the already designed hook(s) are used as primers. In the context of the present invention, for the isolation of said labeled phages (after said viral tagging with a label) from a sample, A. baumannii bacterial cells as target bacteria are applied, whereby such labeled phages get into contact with said target cells by binding to receptors on the surface of A. baumannii.

The term “nucleic acid molecule”, “nucleic acid strand” or the like can be used interchangeably with the term “nucleic acid” and means a polymer comprising one, two, or more nucleotides, e.g., single- or double stranded. Generally, nucleic acids may comprise inter a/ia DNA molecules, RNA molecules, oligonucleotide thiophosphates, substituted ribo-oligonucleotides or PNA molecules. Furthermore, such term may refer to DNA or RNA or hybrids thereof or any modification thereof that is known in the art (see, e.g., U.S. Pat. No. 5,525,711, 4,711,955, 5,792,608 or EP 302175 for examples of modifications). The nucleic acid may be single- or double-stranded, linear or circular, natural or synthetic, and without any size limitation. For instance, the nucleic acid may be genomic DNA, cDNA, mitochondrial DNA, mRNA, antisense RNA, ribozymal RNA or a DNA encoding such RNAs or chimeroplasts (Gamper, Nucleic Acids Research, 2000, 28, 4332-4339). A nucleic acid described herein may also be a fragment of the nucleic acid. Particularly, such a fragment is a functional fragment. Examples for such functional fragments are nucleic acids which can serve as primers.

Such nucleic acid also comprising a nucleic acid sequence as known to a skilled person is part of the phage genome, and hybridizes under stringent conditions to a signature sequence or its complementary sequence as defined elsewhere herein. In this context, the term “being part of its genome” means that the nucleic acid (nucleic acid comprising a nucleic acid sequence) of the isolated phages belongs to the phage genome/is (integrated into) phage genome. Such phage genome (DNA or RNA), which refers to the totality of the genetic information of a phage, is comprised in the head of the phage and is then injected into the target bacteria (A. baumannii in the invention) for replication of its genome within said host cell. Thus, such nucleic acid can also be considered as “phage nucleic acid”. Such phage nucleic acid comprising a nucleic acid sequence may refer to a phage nucleic acid comprising a nucleic acid sequence, which is at least 60%, at least 70%, at least 80%, at least 90%, at least 95%, at least 98%, at least 99% or 100% identical to the nucleic acid sequence encoding the amino acid sequence as depicted in any one selected from the group consisting of SEQ ID NOs: 1-15, such as at least 60%, at least 70%, at least 80%, at least 90%, at least 95%, at least 98%, at least 99% or 100% identical to the nucleic acid sequence encoding the amino acid sequence as depicted in SEQ ID NO: 1; at least 60%, at least 70%, at least 80%, at least 90%, at least 95%, at least 98%, at least 99% or 100% identical to the nucleic acid sequence encoding the amino acid sequence as depicted in SEQ ID NO: 2; at least 60%, at least 70%, at least 80%, at least 90%, at least 95%, at least 98%, at least 99% or 100% identical to the nucleic acid sequence encoding the amino acid sequence as depicted in SEQ ID NO: 3; at least 60%, at least 70%, at least 80%, at least 90%, at least 95%, at least 98%, at least 99% or 100% identical to the nucleic acid sequence encoding the amino acid sequence as depicted in SEQ ID NO: 4; at least 60%, at least 70%, at least 80%, at least 90%, at least 95%, at least 98%, at least 99% or 100% identical to the nucleic acid sequence encoding the amino acid sequence as depicted in SEQ ID NO: 5; at least 60%, at least 70%, at least 80%, at least 90%, at least 95%, at least 98%, at least 99% or 100% identical to the nucleic acid sequence encoding the amino acid sequence as depicted in SEQ ID NO: 6, at least 60%, at least 70%, at least 80%, at least 90%, at least 95%, at least 98%, at least 99% or 100% identical to the nucleic acid sequence encoding the amino acid sequence as depicted in SEQ ID NO: 7; at least 60%, at least 70%, at least 80%, at least 90%, at least 95%, at least 98%, at least 99% or 100% identical to the nucleic acid sequence encoding the amino acid sequence as depicted in SEQ ID NO: 8; at least 60%, at least 70%, at least 80%, at least 90%, at least 95%, at least 98%, at least 99% or 100% identical to the nucleic acid sequence encoding the amino acid sequence as depicted in SEQ ID NO: 9; at least 60%, at least 70%, at least 80%, at least 90%, at least 95%, at least 98%, at least 99% or 100% identical to the nucleic acid sequence encoding the amino acid sequence as depicted in SEQ ID NO: 10; at least 60%, at least 70%, at least 80%, at least 90%, at least 95%, at least 98%, at least 99% or 100% identical to the nucleic acid sequence encoding the amino acid sequence as depicted in SEQ ID NO: 11; at least 60%, at least 70%, at least 80%, at least 90%, at least 95%, at least 98%, at least 99% or 100% identical to the nucleic acid sequence encoding the amino acid sequence as depicted in SEQ ID NO: 12; at least 60%, at least 70%, at least 80%, at least 90%, at least 95%, at least 98%, at least 99% or 100% identical to the nucleic acid sequence encoding the amino acid sequence as depicted in SEQ ID NO: 13; at least 60%, at least 70%, at least 80%, at least 90%, at least 95%, at least 98%, at least 99% or 100% identical to the nucleic acid sequence encoding the amino acid sequence as depicted in SEQ ID NO: 14; and/or at least 60%, at least 70%, at least 80%, at least 90%, at least 95%, at least 98%, at least 99% or 100% identical to the nucleic acid sequence encoding the amino acid sequence as depicted in SEQ ID NO: 15.

The term “nucleotide sequence” as known to a person skilled in the art can also be used interchangeably with the term “nucleic acid sequence”.

By “identity” or “sequence identity” is meant a property of sequences that measures their similarity or relationship. The term “sequence identity” or “identity” as used in the present invention means the percentage of pair-wise identical residues—following (homology) alignment of a sequence—in the present invention the phage nucleic acid sequence—with a sequence in question (the nucleic acid sequence encoding the amino acid sequence as depicted in any one selected from the group consisting of SEQ ID NOs: 1-15) with respect to the number of residues in the longer of these two sequences. Identity is measured by dividing the number of identical residues by the total number of residues and multiplying the product by 100. The percentage of sequence identity can, for example, be determined herein using the program BLASTP, version blastp 2.2.5 (Nov. 16, 2002; cf. Altschul, S. F. et al. (1997) Nucl. Acids Res. 25, 3389-3402).

The level of identity between two or more sequences (e.g., nucleic acid sequences or amino acid sequences) can be easily determined by methods known in the art, e.g., by BLAST analysis. Generally, in context with the present invention, if two sequences (e.g., nucleic acid sequences or amino acid sequences) to be compared by, e.g., sequence comparisons differ in identity, then the term “identity” may refer to the shorter sequence and that part of the longer sequence that matches said shorter sequence. Therefore, when the sequences which are compared do not have the same length, the degree of identity may preferably either refer to the percentage of nucleotide residues in the shorter sequence which are identical to nucleotide residues in the longer sequence or to the percentage of nucleotides in the longer sequence which are identical to nucleotide sequence in the shorter sequence. In this context, the skilled person is readily in the position to determine that part of a longer sequence that matches the shorter sequence. Furthermore, as used herein, identity levels of nucleic acid sequences or amino acid sequences may refer to the entire length of the respective sequence and is preferably assessed pair-wise, wherein each gap is to be counted as one mismatch. These definitions for sequence comparisons (e.g., establishment of “identity” values) are to be applied for all sequences described and disclosed herein.

Moreover, the term “identity” or “identical” as used herein means that there is a functional and/or structural equivalence between the corresponding sequences. Nucleic acid/amino acid sequences having the given identity levels to the herein-described particular nucleic acid/amino acid sequences may represent derivatives/variants of these sequences which, preferably, have the same biological function. They may be either naturally occurring variations, for instance sequences from other varieties, species, etc., or mutations, and said mutations may have formed naturally or may have been produced by deliberate mutagenesis. Furthermore, the variations may be synthetically produced sequences. The variants may be naturally occurring variants or synthetically produced variants, or variants produced by recombinant DNA techniques. Deviations from the above-described nucleic acid sequences may have been produced, e.g., by deletion, substitution, addition, insertion and/or recombination. The term “addition” refers to adding a nucleic acid residue/amino acid to the beginning or end of the given sequence, whereas “insertion” refers to inserting a nucleic acid residue/amino acid within a given sequence. The term “deletion” refers to deleting or removal of a nucleic acid residue or amino acid residue in a given sequence. The term “substitution” refers to the replacement of a nucleic acid residue/amino acid residue in a given sequence. Again, these definitions as used here apply, mutatis mutandis, for all sequences provided and described herein.

The term “hybridize” “hybridizing” refers to “bind” or “binding”, or “being complementary to”, e.g. with regard to the phages to be isolated from an A. baumannii phage colony by applying such hook(s) as defined herein where the phages hybridize to. In detail, the nucleic acid (comprising a nucleic acid sequence) within the phage genome binds to the defined hook, namely a signature sequence or its complementary sequence comprising a nucleic acid sequence which encodes an amino acid sequence as depicted in any one of SEQ ID NOs: 1-15. Such term may also comprise hybridizing to at least a portion of the signature sequence or its complementary sequence as defined herein, which means that said phage nucleic acid hybridizes or is complementary to at least about 3, 4, 5, 6, 7, 8, 9, 20, 11, 12, 13, 14 or 15 (preferably consecutive) nucleotides of the nucleotide sequence comprised by the signature sequence or its complementary sequence encoding any one of the amino acid sequence as shown in SEQ ID NOs: 1-15. That is, for example, in accordance with this embodiment of the present invention, the nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 1; the phage nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 2; the phage nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 3; the phage nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 4; the phage nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 5; the phage nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 6; the phage nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 7; the phage nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 8; the phage nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 9; the phage nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 10; the phage nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 11; the phage nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 12; the phage nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 13; the phage nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 14; and the phage nucleic acid being part of the phage genome hybridizes under stringent conditions to at least about 3, 4, 5, 6, 7, 8, 9, 20, 11, 12, 13, 14 or 15 (preferably consecutive) nucleotides of the nucleotide sequence comprised by the signature sequence or its complementary sequence encoding an amino acid sequence as depicted in SEQ ID NO: 15.

The term “hybridization”, “hybridizing” or “hybridizes” as used herein in context of nucleic acid molecules/DNA or RNA sequences may relate to hybridizations under stringent or non-stringent conditions. If not further specified, the conditions are preferably stringent. Said hybridization conditions may be established according to conventional protocols described, for example, in Sambrook, Russell “Molecular Cloning, A Laboratory Manual”, Cold Spring Harbor Laboratory, N. Y (2001); Current Protocols in Molecular Biology, Update May 9, 2012, Print ISSN: 1934-3639, Online ISSN: 1934-3647; Ausubel, “Current Protocols in Molecular Biology”, Green Publishing Associates and Wiley Interscience, N. Y (1989), or Higgins and Hames (Eds.) “Nucleic acid hybridization, a practical approach” IRL Press Oxford, Washington DC, (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65 °C. Non-stringent hybridization conditions for the detection of homologous or not exactly complementary sequences may be set at 6×SSC, 1% SDS at 65 °C. As is well known, the length of the probe and the composition of the nucleic acid to be determined constitute further parameters of the hybridization conditions. Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. In accordance to the invention described herein, low stringent hybridization conditions for the detection of homologous or not exactly complementary sequences may, for example, be set at 6×SSC, 1% SDS at 65 °C. As is well known, the length of the probe and the composition of the nucleic acid to be determined constitute further parameters of the hybridization conditions.

Hybridizing nucleic acid molecules also comprise fragments of the above described molecules. Such fragments may represent nucleic acid molecules which encode for a functional hook as described herein or a functional fragment thereof. Furthermore, nucleic acid molecules which hybridize with any of the aforementioned nucleic acid molecules also include complementary fragments, derivatives and variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms complementary or complementarity refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions, which depend upon binding between nucleic acids strands. The term “hybridizing nucleic acids/sequences” preferably refers to sequences which display a sequence identity of at least 60%, more preferably at least 65%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98% more preferably at least 99%, more preferably at least 99.5%, and most preferably 100% identity with a nucleic acid/nucleic acid sequence as described herein, e.g. “hybridize” or “hybridizing” as used herein preferably refers to a nucleic acid being part of its genome of the isolated phages as defined herein, wherein the nucleic acid comprises a nucleic acid sequence, which is at least 60%, at least 65%, at least 70%, at least 75%, at least 80%, at least 85%, at least 90%, at least 95%, at least 96%, at least 97%, at least 98%, at least 99%, at least 99.5%, or 100% identical to the nucleic acid sequence encoding the amino acid sequence as depicted in any one of SEQ ID NOs: 1-15, preferably as depicted in SEQ ID NO: 1 and 11.

The term “signature sequence or its complementary sequence hereto” may also refer to a nucleic acid as defined elsewhere herein and is called signature sequence due to the fact that it refers to the particular hook the A. baumannii phages within the composition of the invention hybridize to and which can thus be used for targeted A. baumannii phage isolation. In such isolation such hook may act as a primer within qPCR. Such hook (signature sequence or its complementary sequence hereto) is further defined as a nucleic acid comprising a nucleic acid sequence as defined herein encoding an amino acid sequence as depicted in any one selected from the group consisting of SEQ ID NOs: 1-15. “Complementary” means in this context that the complementary sequence to the signature sequence has the base on each position in the complementary (i.e. A to T, C to G) and in the reverse order as the signature sequence as defined above.

Each signature sequence of its complementary sequence hereto as defined herein encoding an amino acid sequence as depicted in any one selected from the group consisting of SEQ ID NOs: 1-15 refers to a different “hook” which was designed for different phages after metagenomic sequencing. For example, if the hook refers to the signature sequence encoding SEQ ID NO: 1 (which refers to a metallo-phosphoesterase), the composition of the invention may comprise isolated phages (the same phages or different phages) which hybridize to such particular hook. The same applies to the other hooks as defined herein (see any one of SEQ ID NOs: 2-15). If the hook refers to a combination of hooks (for example SEQ ID NOs: 1 and 2), then the composition of the invention may comprise isolated phages (again same phages or different phages) which hybridize with the hook as defined by SEQ ID NO: 1 and with the other hook as defined by SEQ ID NO: 2 (which refers to a HNH endonuclease) or even when different phages are comprised in the composition, some bind to SEQ ID NO: 1, others to SEQ ID NO: 2. Which different A. baumannii phages may thus be comprised by the composition of the invention (meaning which A. baumannii phages may be isolated and then comprised by the composition of the invention), is defined by which hook is applied to which the phages hybridize to—however each phage comprised by the composition is capable of infecting bacterial cells (bacteria) A. baumannii. This enables a novel composition comprising divergent phages capable of infecting A. baumannii which can be isolated by any one of the defined hooks.

In general, infecting bacteria, such as A. baumannii by phages means that such phages either break open (lyses) the bacterial cells, such as A. baumannii, immediately by using e.g. polysaccharide-degrading enzymes which enzymatically degrade the capsular outer layer of such bacteria and thus destroys such bacteria after immediate replication within said bacteria (if it is a lytic phage) or do not lyse the host cells, such as bacteria cells A. baumannii, immediately, but its viral genome integrates with host DNA and replicates along with it before then initiating the reproductive cycle, resulting in lysis of the host cell (if it is a lysogenic phage). In the context of the invention, the phages infecting A. baumannii and which hybridize to the defined hook(s) may refer to lytic phages. To inject a host cell, such as A. baumannii, the phages bind to specific receptors on the surface of A. baumannii such as, but not limited to, outer membrane proteins, polysaccharides, lipopolysaccharides (LPS) and carbohydrate moieties. Thereby, the phages specifically infect only those bacteria, in the case of the invention A. baumannii, bearing certain receptors on their surface to which the phages bind to.

The present invention may thus comprise the composition as defined herein, wherein said composition has bactericidal activity against said A. baumannii. Meaning by applying such composition, e.g. comprised within a pharmaceutical composition, such composition comprising the particular phages is able to take action against the bacterial cell A. baumannii by infecting said bacteria as defined herein, thereby preventing the development of Acinetobacter resistance and thus resulting in an efficient bacterial removal of A. baumannii.

Such A. baumannii can refer to an antimicrobial resistance (AMR) bacterial cell. In this context, as used herein unless specified otherwise, “antimicrobial resistance” may be used interchangeably with “drug resistance” and comprises resistance to at least antibiotic agents known in the art, i.e. the ability of a bacterial cell to resist the effects of a drug or agent that could otherwise successfully treat said bacterial cell. A. baumannii may acquire AMR genes mediating resistance mostly to aminoglycosides, cephalosporins, carbapenems, sulfonamides, tetracyclines and macrolides (Wareth et al. 202, BMC Micobiol 10;21(1):210). The term “antimicrobial resistance” as used herein thus also comprises the term “antibiotic resistance” as known in the art. The present invention may therefore also envisage the composition as defined herein, wherein the A. baumannii bacteria are AMR bacteria.

In a most preferred embodiment of the present invention, the invention relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 1, wherein the phages are capable of infecting A. baumannii. Alternatively, the invention also relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 2, wherein the phages are capable of infecting A. baumannii. Alternatively, the invention also relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 3, wherein the phages are capable of infecting A. baumannii. Alternatively, the invention also relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 4, wherein the phages are capable of infecting A. baumannii. Alternatively, the invention also relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 5, wherein the phages are capable of infecting A. baumannii. Alternatively, the invention also relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 6, wherein the phages are capable of infecting A. baumannii. Alternatively, the invention also relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 7, wherein the phages are capable of infecting A. baumannii. Alternatively, the invention also relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 8, wherein the phages are capable of infecting A. baumannii. Alternatively, the invention also relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 9, wherein the phages are capable of infecting A. baumannii. Alternatively, the invention also relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 10, wherein the phages are capable of infecting A. baumannii. Alternatively, the invention also relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 11, wherein the phages are capable of infecting A. baumannii. Alternatively, the invention also relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 12, wherein the phages are capable of infecting A. baumannii. Alternatively, the invention also relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 13, wherein the phages are capable of infecting A. baumannii. Alternatively, the invention also relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 14, wherein the phages are capable of infecting A. baumannii. Alternatively, the invention also relates to a composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 15, wherein the phages are capable of infecting A. baumannii.

In a further embodiment, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 2-15, wherein the phages are capable of infecting A. baumannii. Such phages within the composition may refer to the same phages, wherein each phage hybridizes to each hook (e.g. encoding SEQ ID NOs: 1 and 2) as well as to different phages, wherein some phages hybridize to the hook encoding SEQ ID NO: 1 and others hybridize to the hook encoding any one of SEQ ID NOs: 2-15. This explanation can be applied to all further embodiments disclosed below, also for embodiments concerning the hybridization to more than two hooks. In a further embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2, wherein the phages are capable of infecting A. baumannii. In a further embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3, wherein the phages are capable of infecting A. baumannii. In a further embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 4, wherein the phages are capable of infecting A. baumannii. In a further embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 5, wherein the phages are capable of infecting A. baumannii. In a further embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 6, wherein the phages are capable of infecting A. baumannii. In a further embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 7, wherein the phages are capable of infecting A. baumannii. In a further embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 8, wherein the phages are capable of infecting A. baumannii. In a further embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 9, wherein the phages are capable of infecting A. baumannii. In a further embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 10, wherein the phages are capable of infecting A. baumannii. In a further embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 11, wherein the phages are capable of infecting A. baumannii. In a further embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 12, wherein the phages are capable of infecting A. baumannii. In a further embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 13, wherein the phages are capable of infecting A. baumannii. In a further embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 14, wherein the phages are capable of infecting A. baumannii. In a further embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 15, wherein the phages are capable of infecting A. baumannii.

The same also applies to embodiments which refer to a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 1 and 3-15, wherein the phages are capable of infecting A. baumannii.

The same also applies to embodiments which refer to a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 1-2 and 4-15, wherein the phages are capable of infecting A. baumannii.

In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 3-15, wherein the phages are capable of infecting A. baumannii. In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3, wherein the phages are capable of infecting A. baumannii. In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 4, wherein the phages are capable of infecting A. baumannii. In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 5, wherein the phages are capable of infecting A. baumannii. In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 6, wherein the phages are capable of infecting A. baumannii. In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 7, wherein the phages are capable of infecting A. baumannii. In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 8, wherein the phages are capable of infecting A. baumannii. In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 9, wherein the phages are capable of infecting A. baumannii. In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 10, wherein the phages are capable of infecting A. baumannii. In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 11, wherein the phages are capable of infecting A. baumannii. In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 12, wherein the phages are capable of infecting A. baumannii. In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 13, wherein the phages are capable of infecting A. baumannii. In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 14, wherein the phages are capable of infecting A. baumannii. In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2 and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 15, wherein the phages are capable of infecting A. baumannii.

In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3, and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 4-15, wherein the phages are capable of infecting A. baumannii.

In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 4, and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 5-15, wherein the phages are capable of infecting A. baumannii.

In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 4, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 5, and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 6-15, wherein the phages are capable of infecting A. baumannii.

In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 4, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 5, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 6, and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 7-15, wherein the phages are capable of infecting A. baumannii.

In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 4, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 5, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 6, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 7, and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 8-15, wherein the phages are capable of infecting A. baumannii.

In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 4, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 5, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 6, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 7, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 8, and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 9-15, wherein the phages are capable of infecting A. baumannii.

In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 4, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 5, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 6, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 7, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 8, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 9, and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 10-15, wherein the phages are capable of infecting A. baumannii.

In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 4, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 5, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 6, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 7, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 8, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 9, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 10, and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 11-15, wherein the phages are capable of infecting A. baumannii.

In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 4, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 5, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 6, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 7, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 8, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 9, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 10, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 11, and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 12-15, wherein the phages are capable of infecting A. baumannii.

In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 4, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 5, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 6, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 7, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 8, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 9, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 10, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 11, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 12, and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 13-15, wherein the phages are capable of infecting A. baumannii.

In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 4, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 5, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 6, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 7, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 8, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 9, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 10, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 11, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 12, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 13, and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in any one selected from the group consisting of SEQ ID NOs: 14-15, wherein the phages are capable of infecting A. baumannii.

In another embodiment of the invention, the invention comprises a composition comprising isolated phages, wherein the (same or different) phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and wherein the composition further comprises (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 2, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 3, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 4, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 5, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 6, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 7, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 8, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 9, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 10, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 11, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 12, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 13, further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 14, and further comprising (the same or different) phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 15, wherein the phages are capable of infecting A. baumannii.

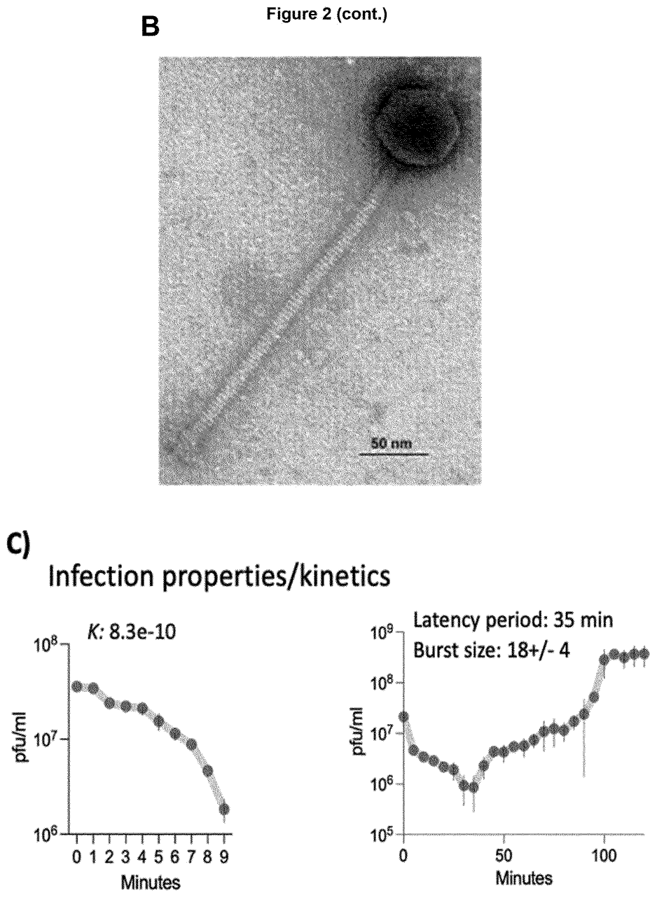

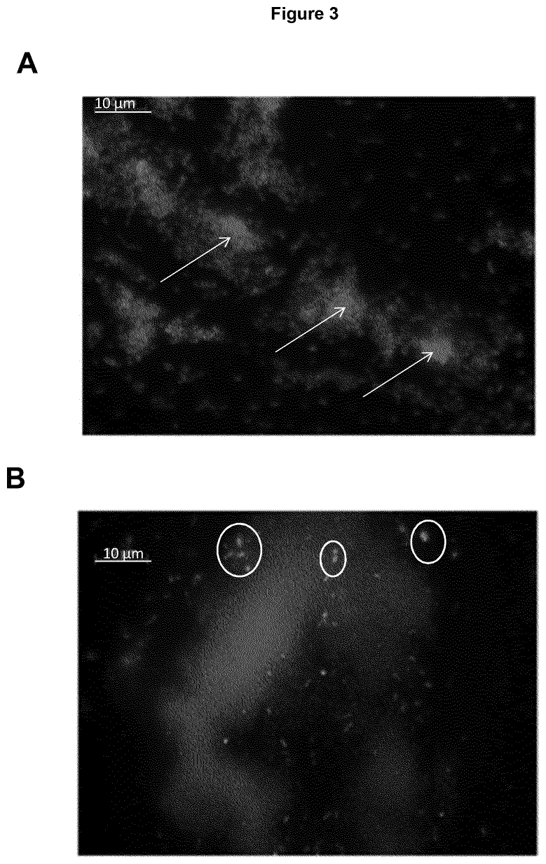

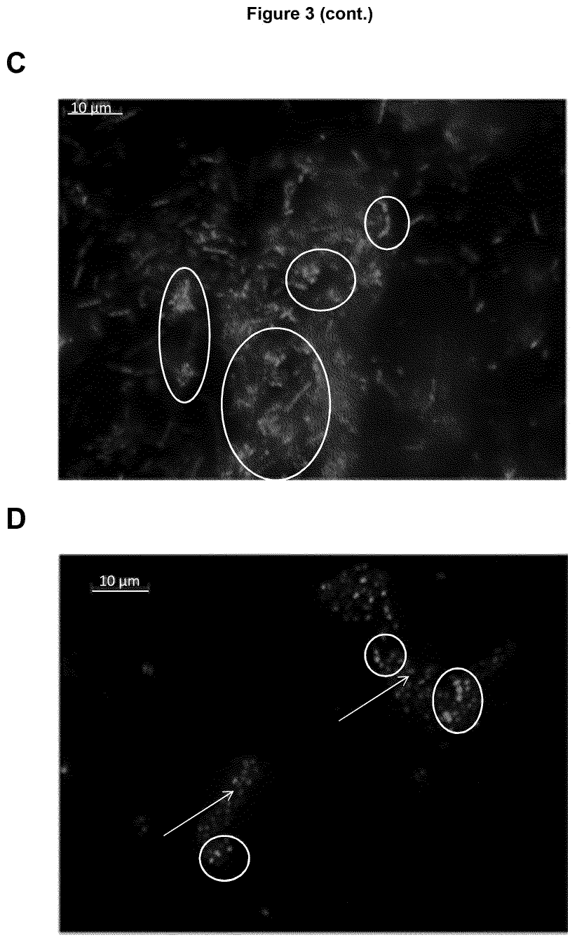

In a most preferred embodiment of the invention, the composition as defined elsewhere herein comprises one or more phages selected from the group consisting of vB_AbS_HMGU, vB_AbM_HMGU and vB_AbA_HMGU (see FIG. 2), preferably the combination of vB_AbM_HMGU and vB_AbA_HMGU which showed a synergetic effect when used in combination against A. baumanii thus resulting in a higher efficiency compared to the corresponding single phage (see FIG. 3). Said particular phages have been named according to the ICTV guidelines for naming phages. The A. baumannii phage labeled “AbA” binds to five signature sequences (or complementary sequences hereto) each comprising a nucleic acid sequence, wherein the first one encodes an amino acid sequence as depicted in SEQ ID NO: 1 (refers to a metallo-phosphoesterase), the second one encodes an amino acid sequence as depicted in SEQ ID NO: 2 (refers to a HNH endonuclease), the third one encodes an amino acid sequence as depicted in SEQ ID NO: 3 (refers to a terminase), the fourth one encodes an amino acid sequence as depicted in SEQ ID NO: 4 (refers to endolysin), the fifth one encodes an amino acid sequence as depicted in SEQ ID NO: 5 (refers to a head-tail adaptor), the signature sequence (or complementary sequence hereto) encoding an amino acid sequence as depicted in SEQ ID NO: 1 as the hook with regard to all 15 hooks depicted herein where the most phages hybridize to—among others AbA. The A. baumannii phage labeled “AbS” binds to other five signature sequences (or complementary sequences hereto) each comprising a nucleic acid sequence, wherein the first one encodes an amino acid sequence as depicted in SEQ ID NO: 6 (refers to endolysin), the second one encodes an amino acid sequence as depicted in SEQ ID NO: 7 (refers to a co-chaperonin GroES), the third one encodes an amino acid sequence as depicted in SEQ ID NO: 8 (refers to a SbcD-like subunit of palindrome specific endonuclease), the fourth one encodes an amino acid sequence as depicted in SEQ ID NO: 9 (refers to a thymidylate synthase), the fifth one encodes an amino acid sequence as depicted in SEQ ID NO: 10 (refers to a ribonucleoside-diphosphate reductase). The A. baumannii phage labeled “AbM” binds to other five signature sequences (or complementary sequences hereto) each comprising a nucleic acid sequence, wherein the first one encodes an amino acid sequence as depicted in SEQ ID NO: 11 (refers to a thymidylate synthase), the second one encodes an amino acid sequence as depicted in SEQ ID NO: 12 (refers to a dCMP deaminase), the third one encodes an amino acid sequence as depicted in SEQ ID NO: 13 (refers to a phosphoesterase), the fourth one encodes an amino acid sequence as depicted in SEQ ID NO: 14 (refers to a phosphatase), the fifth one encodes an amino acid sequence as depicted in SEQ ID NO: 15 (refers to a endolysin), the signature sequence (or complementary sequence hereto) encoding an amino acid sequence as depicted in SEQ ID NO: 11 as the hook with regard to the hooks depicted in SEQ ID NOs: 11-15 where the most phages hybridize to—among others AbM. All of the abovementioned proteins as depicted in SEQ ID NOs: 1-15 have an important role in the bacteria lysing capacity of the phages.

### Pharmaceutical Composition

The present invention also relates to a pharmaceutical composition comprising the novel composition including the isolated phages as defined herein. Thus, such novel composition of the invention can also be a pharmaceutical composition. Such pharmaceutical composition may further comprise one or more of the ingredients (such as at least one excipient) as mentioned above for the composition of the invention. If at least one excipient is further comprised by the pharmaceutical composition, such excipient refers to at least one pharmaceutically acceptable excipient. Suitable pharmaceutical excipients are further described in Remington's Pharmaceutical Sciences, Mack Publishing Company, a standard reference text in this field. Said pharmaceutically acceptable excipient includes any excipient that does not itself elicit an adverse reaction harmful to the subject receiving the pharmaceutical composition. If the composition of the invention additionally comprises at least one pharmaceutically acceptable excipient, said composition refers to a pharmaceutical composition. Said pharmaceutical composition is thus used herein for therapeutic purposes. Moreover, the present invention relates to the use of said composition as disclosed herein for the preparation of a pharmaceutical composition.

In accordance with the present invention, the term “pharmaceutical composition” relates to a composition for administration to a subject as defined herein, preferably a human. Pharmaceutical compositions or formulations are usually in such a form as to allow the biological activity of the active ingredient to be effective and may therefore be administered to a subject for therapeutic use as described herein. The pharmaceutical composition can be administered in a therapeutically effective amount as defined elsewhere by inhalation, injection, infusion, or orally. The administration of said pharmaceutical composition as defined elsewhere herein may be performed intraperitoneally, intravenously, intraarterially, subcutaneously, intramuscularly, parenterally, transdermally, intraluminally, intrathecally, intranasally and/or directly into tissue due to the fact that infection with A. baumannii can take place in different parts of the body/in multiple body sites of the subject. The pharmaceutical compositions can be administered to the subject at a suitable dose. The dosage regimen will be determined by the attending physician and by clinical factors. As is well known in the medical arts, dosages for any one patient depend upon many factors, including the patient's size, body surface area, age, the particular compound to be administered, sex, time and route of administration, general health, and other drugs being administered concurrently.

Suitable excipients are typically large, slowly metabolized macromolecules such as proteins, polysaccharides, polylactic acids, polyglycolic acids, polymeric amino acids, amino acid copolymers and lipid aggregates such as e.g. oil droplets or liposomes. The excipient used in combination with the (pharmaceutical) composition of the present invention may be water-based and forms an aqueous solution. An oil-based excipient solution is an alternative to the aqueous excipient solution.

Also, the pharmaceutical composition as defined herein may further comprise one or more adjuvants. The term “adjuvant” is used according to its well-known meaning in connection with pharmaceutical compositions. Specifically, an adjuvant is an immunological agent that modifies, preferably enhances, the effect of such composition while having few, if any, desired immunogenic effects on the immune system when given per se. Suitable adjuvants can be inorganic adjuvants such as, e.g., aluminium salts (e.g., aluminium phosphate, aluminium hydroxide), monophosphoryl lipid A, or organic adjuvants such as squalene or oil-based adjuvants, as well as virosomes.

Definitions made herein for the composition of the invention also apply mutatis mutandis to the pharmaceutical composition.

### In Vivo Therapeutic Applications

The present invention further refers to said composition or said pharmaceutical composition comprising the novel composition of the invention as defined elsewhere herein for use as a medicament. Hence, said composition or said pharmaceutical composition comprising the novel composition of the invention as defined elsewhere herein can also be used for therapy, i.e. treating or preventing an infection of a subject in need thereof with A. baumannii. Accordingly, said composition or said pharmaceutical composition comprising the novel composition of the invention as defined elsewhere herein is particularly suitable for use in a method of preventing or treating an infection with A. baumannii of a subject in need thereof.

As such the term “treat”, “treating” or “treatment” as used herein means to reduce (slow down (lessen)), stabilize or inhibit or at least partially alleviate or abrogate the progression of the symptoms associated with the respective disease, such as an infection of a subject with A. baumannii. Thus, it includes the administration of said (pharmaceutical) composition, preferably in the form of a medicament, to a subject, defined elsewhere herein. Those in need of treatment include those already suffering from the disease, here an infection with A. baumannii as described elsewhere herein. Preferably, a treatment reduces (slows down (lessens)), stabilizes, or inhibits or at least partially alleviates or abrogates progression of a symptom that is associated with the presence and/or progression of such disease (such infection). “Treat”, “treating”, or “treatment” refers to a therapeutic treatment. In particular, in the context of the present invention, treating or treatment refers to an improvement of the symptom(s) that is associated with said infection with A. baumannii as defined elsewhere herein in a subject in need thereof. In this context, the term “treat”, “treating” or “treatment” refers to an anti-bacterial therapy that directly attacks said bacterial cells A. baumannii the subject has been infected with, by the phages then infecting said bacterial cells A. baumannii (is then the host cell of the phages for replication) as defined herein. Thus the (pharmaceutical) composition as defined herein may also be used as anti-bacterial therapeutic.

The term “prevent”, “preventing”, “prevention” as used herein refers to prophylactic or preventative measures, wherein the subject is to prevent an abnormal, including pathologic, condition in the organism which would then lead to the defined disease, namely said infection with A. baumannii as defined herein. In other words, said terms refer to a medical procedure whose purpose is to prevent such disease meaning inhibiting that a subject will likely suffer from any future infection with A. baumannii as defined herein. Meaning the compositions comprising said phages will be given preventive to the subject not yet being infected with A. baumannii. If the subject is then contacted and then infected with A. baumannii, the phages comprised in the already administered composition can then attack the bacteria right away thereby preventing a full infection with A. baumannii. As used herein, such terms also refer to the reduction in the risk of acquiring or developing such infection with A. baumannii in a patient. Those in need of the prevention include those prone to having the disease, such as the infection with A. baumannii. In other words, those who are of a risk to develop such infection and will thus probably suffer from it in the near future. Subjects suffering from diabetes or COPD are more likely to get such infection. Also other factors such as having a weak immune system, poor hygiene, being in the intensive care unit or being on a ventilator (breathy machine)/having a catheter for quite some time, recently having a surgery or having open wounds caused by an accident or an injury contribute to a higher risk of suffering from such infection. Thus, the (pharmaceutical) composition as defined herein may also be used as a prophylaxis, not only as a therapeutic agent for a subject as defined herein that has already been infected with A. baumannii.

An infection with A. baumannii may refer to any infection which occurs in different parts of the body/in multiple body sites of the subject, including but not limited to the lung, the blood, the brain, the urinary tract, and also wounds. Exemplarily, an infection with A. baumannii may be any one of a nosocomial pneumonia, an inflammatory lung infection, a blood infection (due to the fact for example that the bacteria enter through a catheter placed in a subject's vein), meningitis (after surgery on the brain or spine), urinary tract infection (infection of the kidneys, ureters or the bladder), or a skin or wound infection. Such bacteria can easily spread by direct contact with a subject already infected with A. baumannii, since it is found on the skin or also by contact with food, water or soil which also comprises said bacteria. As mentioned elsewhere herein, such bacteria are often susceptible or can be even resistant to many antibiotics which makes treatment of an infection with A. baumannii so difficult. For the purpose of distinguishing resistant from susceptible bacteria, the FEEDAP Panel defines microbiological cut-off values. Microbiological cut-off values (mg/L) are set by studying the distribution of minimum inhibitory concentrations (MICs) of the chosen antimicrobials in bacterial populations belonging to a single taxonomical unit (species or genus). The part of the population that clearly deviates from the normal susceptible populations is categorised as resistant. In particular, a bacterial cell is defined as susceptible when it is inhibited at a concentration of a specific antimicrobial equal or lower than the established cut-off value (S≤x mg/L) defined by FEEDAP Panel. A bacterial cell is defined as resistant when it is not inhibited at a concentration of a specific antimicrobial higher than the established cut-off value (R>x mg/L) defined by FEEDAP Panel. The present invention may therefore also comprise the composition of the invention or the pharmaceutical composition for the use in a method of treating or preventing an infection with A. baumannii as defined herein, wherein the A. baumannii bacteria are AMR bacteria as defined elsewhere herein.

The term “subject” when used herein includes mammalian and non-mammalian subjects. Preferably the subject of the present invention is a mammal, including human, domestic and farm animals, non-human primates, and any other animal that has mammary tissue. In some embodiments the mammal is a mouse or a rat. In some embodiments the mammal is a guinea pig or a rabbit. In some embodiments the mammal is a cat. In some embodiments the mammal is a dog. In some embodiments the mammal is a monkey. In some embodiments the mammal is a horse. In preferred embodiment the mammal of the present invention is a human, most preferred an adult. Where the subject is a living human who may receive treatment for such infection as described herein, it is also addressed as a “patient”. In some embodiments the subject of the present invention suffers from said infection as described herein. The term “suffering” as used herein means that the subject is not any more a healthy subject. The term “healthy” means that the respective subject has no obvious or noticeable hallmarks or symptoms of the respective infection. This further means that the subject suffering from said infection is a subject “in need” of the respective treatment with the (pharmaceutical) composition as defined herein.

Such (pharmaceutical) composition is generally administered to the subject in a therapeutically effective amount. Said therapeutically effective amount is sufficient to inhibit or alleviate the symptoms of said infection. By “therapeutic effect” or “therapeutically effective” is meant that the conjugate for use will elicit the biological or medical response of a tissue, system, animal or human that is being sought by the researcher, veterinarian, medical doctor or other clinician. The term “therapeutically effective” further refers to the inhibition of factors causing or contributing to the infection. The term “therapeutically effective amount” includes that the amount of the composition when administered is sufficient to significantly improve the progression of the infection being treated or to prevent development of said infection. According to a preferred embodiment, the therapeutic effective amount is sufficient to alleviate or heal said infection as defined herein.

The therapeutically effective amount will vary depending on the (pharmaceutical) composition of the present invention, the infection with A. baumannii and its severity and on individual factors of the subject and/or also how the administration works. Therefore, the (pharmaceutical) composition of the present invention will not in all cases turn out to be therapeutically effective, because the method disclosed herein cannot provide a 100% safe prediction whether or not a subject may be responsive to the (pharmaceutical) composition, since individual factors are involved as well. It is to expect that age, body weight, general health, sex, diet, drug interaction and the like may have a general influence as to whether or not the (pharmaceutical) composition for use in the treatment of a subject suffering from said infection will be therapeutically effective.

The term “administering” or “administered” or “administration” used throughout various aspects of the present invention means that the (pharmaceutical) composition as defined herein are given to the respective subject in an appropriate form and dose and using appropriate measures. The administration of the composition according to the present invention can be carried out by any method known in the art.

In alternative embodiments, the (pharmaceutical) composition for the use in the treatment of said infection may also be administered in combination with an additional therapeutic agent (drug). Drugs or therapeutic agents useful in this regard include without limitation drug-like molecules, proteins, peptides, and small molecules. Protein therapeutic agents include, without limitation peptides, enzymes, antibodies, structural proteins, receptors and other cellular or circulating proteins as well as fragments and derivatives thereof, preferably an additional therapeutic agent/drug in the context of the present invention may be a drug for the use in an infection with A. baumannii as described elsewhere herein, especially for combinatorial therapy in said infection. Said combination according to the present invention can be administered as a combined formulation or separate from each other.

The present invention also provides for a method of treating or preventing an infection with A. baumannii in a subject in need thereof, the method comprising administering a therapeutically effective amount of said composition or said pharmaceutical composition to a subject in need thereof. Also comprised herein is the use of said (pharmaceutical) composition as defined herein for the manufacture of a medicament for therapeutic application in an infection with A. baumannii in a subject. The definitions and embodiments made with regard to the first and second medical uses may also be applied, where necessary, in these embodiments as well.

### Hook

When the invention refers to a nucleic acid, which hybridizes under stringent conditions as defined herein to a signature sequence or its complementary sequence comprising a nucleic acid sequence encoding an amino acid sequence as depicted in any one selected from the group consisting of SEQ ID NOs: 1-15 (the “hook(s)” of the invention, see below), said nucleic acid may also comprise a nucleic acid sequence which is at least 60%, at least 70%, at least 80%, at least 90%, at least 95%, at least 98%, at least 99% or 100% identical to the nucleic acid sequence encoding the amino acid sequence as depicted in any one selected from the group consisting of SEQ ID NOs: 1-15. Thus, a nucleic acid, which hybridizes under stringent conditions to a signature sequence or its complementary sequence comprising a nucleic acid sequence encoding an amino acid sequence as depicted in SEQ ID NO: 1, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 1 may also be comprised herein. Also comprised herein, is a nucleic acid, which hybridizes under stringent conditions to a signature sequence or its complementary sequence comprising a nucleic acid sequence encoding an amino acid sequence as depicted in SEQ ID NO: 2, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 2. Further, comprised herein, is a nucleic acid, which hybridizes under stringent conditions to a signature sequence or its complementary sequence comprising a nucleic acid sequence encoding an amino acid sequence as depicted in SEQ ID NO: 3, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 3. Also comprised herein, is a nucleic acid, which hybridizes under stringent conditions to a signature sequence or its complementary sequence comprising a nucleic acid sequence encoding an amino acid sequence as depicted in SEQ ID NO: 4, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 4. Further comprised herein, is a nucleic acid, which hybridizes under stringent conditions to a signature sequence or its complementary sequence comprising a nucleic acid sequence encoding an amino acid sequence as depicted in SEQ ID NO: 5, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 5. Also comprised herein, is a nucleic acid, which hybridizes under stringent conditions to a signature sequence or its complementary sequence comprising a nucleic acid sequence encoding an amino acid sequence as depicted in SEQ ID NO: 6, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 6. Also comprised herein, is a nucleic acid, which hybridizes under stringent conditions to a signature sequence or its complementary sequence comprising a nucleic acid sequence encoding an amino acid sequence as depicted in SEQ ID NO: 7, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 7. Further comprised herein, is a nucleic acid, which hybridizes under stringent conditions to a signature sequence or its complementary sequence comprising a nucleic acid sequence encoding an amino acid sequence as depicted in SEQ ID NO: 8, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 8. Also comprised herein, is a nucleic acid, which hybridizes under stringent conditions to a signature sequence or its complementary sequence comprising a nucleic acid sequence encoding an amino acid sequence as depicted in SEQ ID NO: 9, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 9. Further comprised herein, is a nucleic acid, which hybridizes under stringent conditions to a signature sequence or its complementary sequence comprising a nucleic acid sequence encoding an amino acid sequence as depicted in SEQ ID NO: 10, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 10. Also comprised herein, is a nucleic acid, which hybridizes under stringent conditions to a signature sequence or its complementary sequence comprising a nucleic acid sequence encoding an amino acid sequence as depicted in SEQ ID NO: 11, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 11. Also comprised herein, is a nucleic acid, which hybridizes under stringent conditions to a signature sequence or its complementary sequence comprising a nucleic acid sequence encoding an amino acid sequence as depicted in SEQ ID NO: 12, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 12. Further comprised herein, is a nucleic acid, which hybridizes under stringent conditions to a signature sequence or its complementary sequence comprising a nucleic acid sequence encoding an amino acid sequence as depicted in SEQ ID NO: 13, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 13. Also comprised herein, is a nucleic acid, which hybridizes under stringent conditions to a signature sequence or its complementary sequence comprising a nucleic acid sequence encoding an amino acid sequence as depicted in SEQ ID NO: 14, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 14. Finally, comprised herein, is a nucleic acid, which hybridizes under stringent conditions to a signature sequence or its complementary sequence comprising a nucleic acid sequence encoding an amino acid sequence as depicted in SEQ ID NO: 15, wherein said nucleic acid comprises a nucleic acid sequence which is at least 60%, at least 70%, at least 80%, at least 90%, at least 95%, at least 98%, at least 99% or 100% identical to the nucleic acid sequence encoding the amino acid sequence as depicted in SEQ ID NO: 15.

As mentioned elsewhere herein, such particular hook (“signature sequence or complementary sequence hereto” as defined elsewhere herein with regard to the composition) e.g. which is used for targeted A. baumannii phage isolation, refers to a nucleic acid which comprises a nucleic acid sequence encoding an amino acid sequence as depicted in any one selected from the group consisting of SEQ ID NOs: 1-15, or a complementary sequence thereof. Thus, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in any one selected from the group consisting of SEQ ID NOs: 1-15, or a complementary sequence thereof. In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 1, or a complementary sequence thereof. As explained elsewhere herein, the hook which encodes the metallo-phosphoesterase as defined by SEQ ID NO: 1 is the most abundant protein family within the isolated A. baumannii phages after having analysed the genome of the A. baumannii phages as defined elsewhere herein. Using such designed hook for targeted phage isolation, targets most A. baumannii phages, resulting in the most divergent A. baumannii phage composition.

In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 2, or a complementary sequence thereof. In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 3, or a complementary sequence thereof. In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 4, or a complementary sequence thereof. In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 5, or a complementary sequence thereof. In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 6, or a complementary sequence thereof. In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 7, or a complementary sequence thereof. In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 8, or a complementary sequence thereof. In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 9, or a complementary sequence thereof. In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 10, or a complementary sequence thereof. In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 11, or a complementary sequence thereof. In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 12, or a complementary sequence thereof. In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 13, or a complementary sequence thereof. In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 14, or a complementary sequence thereof. In another embodiment, the invention also comprises as a hook a nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 15, or a complementary sequence thereof.

In this context, said nucleic acid which hybridizes under stringent conditions to a signature sequence or its complementary sequence comprising a nucleic acid sequence encoding an amino acid sequence as depicted in any one selected from the group consisting of SEQ ID NOs: 1-15 as defined herein or said nucleic acid of the hook as defined herein may also be conjugated to a detectable label. It is also comprised herein, that such nucleic acids may be attached to a detectable label or just labeled. A “detectable label” as used herein may refer to a compound capable of targeting said nucleic acids and which can then be detected by any detection methods known to a person skilled in the art, which depends certainly on the label being used. Said label may be selected from the group consisting of a biotin, a tag, a fluorophore, a radioactive phosphate (P³²) or radioactive sulfate (S³⁵), and any active site probe, i.e. a detectable moiety suitable for the labeling and detection of said nucleic acids. The nucleic acids may also be conjugated to a combination of any one of the detectable labels mentioned above. For example, the nucleic acids may be conjugated to a detectable fluorophore and further labeled with a radioactive phosphate.

When the label is a biotin, nucleotides of the nucleic acid sequences comprised by said nucleic acids may be labeled with biotin. When the label is a tag, an oligohistidine-tag (His₆ tag) may be used. In this context, the nucleic acid may be modified with for example nitrilotriacetate (NTA) which has high affinity to a His-tag. When the label is a fluorophore (also called fluorochrome or chromophore) in the present invention it may be any DNA fluorescent dye such as but not limited to Acridine Orange hydrochloride, 7-Aminoactinomycin D, DAPI, Hoechst 3342, Hoechst Janelia Fluor 526, Hoechst Janelia Fluor 646, Nucleic Acid Dye Green I, propidium iodide, or one of a fluorescent dye such as but not limited to Fluorescein (FITC), Alexa Fluor 350, 405, 488, 532, 546, 555, 568, 594, 647, 680, 700, 750, Pacific Blue, Coumarin, Pacific Green, Cy3, Texas Red, PE, PerCP-Cy5, PE-Cy7, Pacific Orange, or a fluorescent protein label such as R-PE or APC, or an expressed fluorescent protein such as CFP, EGFP, GFP or RFP. Preferably, when the label is a fluorophore any DNA fluorescent dye is used. The labeling with such fluorophore (preferably any DNA fluorescent dye) may be performed by labeling the nucleotides of the nucleic acids as it is known to a person skilled in the art. When the label is a radioactive phosphate (P³²) or sulfate (S³⁵), one of the incorporated nucleotides may be radiolabeled on the alpha phosphate position.

Said nucleic acids may be labeled at their 5′ end (DNA 5′ end labeling), their 3′ end (DNA 3′ end labeling), or throughout the nucleic acid depending on the application (for example labeling by PCR). For hybridization as outlined herein, it is preferred to generate high specific activity probes with such label distributed throughout the nucleic acid, through techniques such as nick translation, random priming, by PCR or in vitro transcription using labeled dNTPs or NTPs. The conjugation (attachment) of a label (preferably a fluorophore, even more preferably any DNA fluorescent dye) may be either direct (without any linker) or indirect via a linker. Thus, it is also comprised herein that the nucleic acids may be conjugated via a linker/linked to a detectable label as defined herein. Said conjugation to the detectable label may also be covalently as defined elsewhere herein. Thus, it is also comprised herein that the nucleic acids are directly or indirectly conjugated to a detectable label, preferably covalently. The term “covalently” refers to covalent bonds that are typically formed by the sharing of electron pairs between atoms. In accordance with the present invention and when the term “covalently” is used, a covalent bond is formed between the nucleic acids and the label as defined elsewhere herein by use of a linker of at least 1 amino acid in length as defined above.

### Kit

The present invention also relates to a kit comprising the composition comprising the isolated phages of the present invention or said pharmaceutical composition comprising such composition. Thus, when a kit comprises said (pharmaceutical) composition, said compositions may be provided in a vial or a container, preferably also comprising in said vial or container at least one excipient as defined herein. Further, said kit may be associated with a notice in the form prescribed by a governmental agency regulating the manufacture, use or sale of pharmaceuticals or biological products, reflecting approval by the agency of the manufacture, use or sale of the product for human administration or diagnostics. Said kit may comprise the (pharmaceutical) composition, preferably in a vial or container, in dried form, such as a lyophilized, air-dried, or spray-dried form (in form of a powder), for later reconstitution into a liquid form or other form prior to use. Further, said kit may also comprise the (pharmaceutical) composition, preferably in a vial or container, in a frozen state, being thawed prior to use. Further, said kit may also comprise the (pharmaceutical) composition, preferably in a vial or container, in liquid state.

The present invention also comprises a kit comprising the hook(s) as defined elsewhere herein. Thus, when a kit comprises said hook(s), said hook(s) may be provided in a vial or a container, preferably also comprising in said vial or container at least one excipient as defined herein. Either the kit may provide a vial or container comprising one of the defined hooks, meaning one kit comprises one of the defined hooks, or a kit may comprise at least one of the defined hooks, meaning in one kit there are more than one hooks (at least any one of SEQ ID NOs: 1-15) comprised. With regard to the latter (combination of hooks within a kit), such hooks can either be comprised separately in different vials or containers or comprised all together in one vial or container. Further, said kits may be associated with a notice in the form prescribed by a governmental agency regulating the manufacture, use or sale of pharmaceuticals or biological products, reflecting approval by the agency of the manufacture, use or sale of the product for human administration or diagnostics. Said kits may comprise the hook(s), preferably in a vial or container, in dried form, such as a lyophilized, air-dried, or spray-dried form (in form of a powder), for later reconstitution into a liquid form or other form prior to use. Further, said kits may also comprise the hook(s), preferably in a vial or container, in a frozen state, being thawed prior to use. Further, said kits may also comprise the hook(s), preferably in a vial or container, in liquid state.

Additionally, the kit according to the present invention either comprising the (pharmaceutical) composition or the hook(s) may also comprise a detectable label as defined elsewhere herein. Such label may be selected from the ones as listed above. In some embodiments, said label may also be comprised in the one or more containers or vials of the kit as defined above comprising said (pharmaceutical) composition or in the one or more containers or vials of the kit as defined above comprising said hook(s); or in additional one or more vials or containers of said kits, preferably further comprising in said one or more vials or containers any excipient suitable for said label to be mixed with/contacted with.

In some embodiments, said hook(s) may also be comprised in the same kit which comprises said (pharmaceutical) composition as defined above (with or without said label within the same kit).

### In Vitro Applications

The present invention also relates to a method of preparing a composition comprising isolated phages, the method comprising the following steps of first providing a sample which comprises the phages to be isolated from. Such step may also be optional. Such sample may be any one of a naturally environmental sample, a clinical sample, a wastewater treatment plant sample which includes but is not limited to a sewage water sample, or a hospital waste resources sample. In a next step, said phages are isolated from said sample as defined herein using any one of the defined hooks of the invention. “Targeted isolating” or “targeted isolation” (“hook isolation”) may be carried out by any method known in the art suitable to isolate phages based on selected gene or nucleic acid molecule sequences. Such step may also include a viral tagging (also called phage labeling) step as defined by WO2021/048257 incorporated by references herein resulting in the phages being labeled, wherein the labelling may be selected from the group consisting of fluorescent labelling, antibody labelling and radioactive labelling (preferably fluorescent labelling). Then, target bacteria (in the context of the invention A. baumannii) may be added to said sample comprising the already labeled phages, whereby said labeled phages come into contact with the target bacteria. This may be followed by single-cell sorting of such bacteria-phages into single pairs. As a general example in accordance with the present invention, single phage-bacteria pairs may then be sorted onto solid bacterial lawns, or liquid growth media (e.g. in multi-well plate), and incubated under conditions allowing phage infection take place.

Agar from the infected area (plaque) in the former approach, or aliquots of phage suspensions in the latter approach may then be transferred to a vial (e.g. to 2^{nd} multi-well plates) containing premixed (e.g., barcoded) primer(s), which refer to at least any one of the defined hooks already designed before. Such vial may also comprise besides the primer(s) a PCR Master mix for PCR or qPCR. This results in isolating the particular A. baumannii phages which hybridize to the particular hooks (primers in the qPCR) being used for said targeted phage isolation.

For the targeted phage isolation to be performed, such phages comprise a nucleic acid being part of its genome, which hybridizes under stringent conditions to a signature sequence or its complementary sequence comprising a nucleic acid sequence encoding an amino acid sequence as depicted in any one selected from the group consisting of SEQ ID NOs: 1-15, preferably SEQ ID NO: 1, which refers to the defined hook being used for targeted phage isolation. Alternatively, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 1. In another embodiment, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 2. In a further embodiment, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 3. In another embodiment, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 4. In another embodiment, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 5. In another embodiment, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 6. In another embodiment, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 7. In another embodiment, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 8. In another embodiment, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 9. In another embodiment, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 10. In another embodiment, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 11. In another embodiment, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 12. In another embodiment, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 13. In another embodiment, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 14. In another embodiment, such phages may comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 15.

After the abovementioned targeted phage isolation, the isolated phages are obtained and provided within a composition as defined herein, meaning such isolated phages are then added to a pre-prepared composition as defined herein. The abovementioned can also be applied mutatis mutandis to a method of preparing a pharmaceutical composition as defined elsewhere herein.

Each definition made throughout the invention can also be applied, where necessary, to the method of preparing such (pharmaceutical) composition as well as to a composition comprising isolated phages obtainable or obtained by such method.

### EXAMPLES OF THE INVENTION

### Materials and Methods

Sample collection.

Sewage water was sampled regularly and kept at 4 °C before processing. The samples were used for isolating phages using classical isolation and viral tagging (VT).

### Classical Phage Isolation.

The phages were amplified by mixing 50 ml of wastewater with the same amount of double-strength lysis buffer (LB) and 10 ml of a single Escherichia coli Reference collection (ECOR) strain bacteria cultured overnight. After incubation overnight at 30 °C, 10 ml of the mixture was centrifuged at 6000×g at +4 °C for 15 minutes and sterile filtered through a 0.45 μm membrane filter. Then, the phage titer was measured in plaque assays. Sterile filtered phage lysates were diluted in SM buffer or LB to five different dilutions (10e5 to 10e9). 100 μl of diluted phage and 200 μl of target bacteria (A. baumannii) were mixed with 2 ml soft agar (SA), spread on pre-warmed LA plates, and incubated overnight at 30 °C. The harvested phages were selected according to their plaque morphology.

### VT Experimental Detail.

### Viral Stain and Wash.

Dyes used to stain viruses, e.g. SYTO 9, SYBR Gold, SYBR Green II and SYBR Safe, were diluted to 50× in TE buffer (10 mM Tris, 1 mM EDTA; pH 8.0) for storage in −20 °C in the dark until dilution to final concentration of 1-5× for preparation of viral particles for VT (cf. Džunková et al. (Nature Microbiology (2019)) for labeling clinical samples). Viral samples were stained with dyes at 30 °C for 30 min for the environmental samples. The ultracentrifugal devices (100 KDa cut-off; Amicon) were pretreated by incubating 1.5 ml of 0.2 μm-filter-sterilized 1% BSA (Bioexpress, UT, cat #E531-1.5ML) in phosphate buffered saline (PBS) for 1 h at room temperature. Stained wastewater samples were washed six times by phage buffer in the pretreated ultracentrifugal devices. 50 μl phage buffer were added back for every 500 ml viral samples and sonicated (VWR Signature Ultrasonic cleaner B1500A-DTH, VWR) for 3 minutes using the settings of 50 W at 42 kHz, resulted a 10-fold concentration of viruses from the original sample. Stained and washed viruses were mixed with bacteria (A. baumannii) at concentrations and ratios desired for flow cytometer analysis, typically 10⁵ cells per ml. VT experiments were done with a negative control, which was prepared identically to the stained and washed virus samples except without viruses; this controlled for free dye creating the appearance of false positive ‘viral tagged cells’.

The concentration of bacterial cells or viral particles to be combined in the VT assay was then adjusted by saline solution (SS, 0.9% NaCl) to obtain a ratio of ~100:1 to 1:100 (bacteria to phage), where >1.000 cells/see were processed by FACS. Six 200 μl aliquots of the washed and diluted cell preparation were used as: 1) unstained bacterial cells mixed with SS, 2) stained bacterial cells mixed with SS, 3) unstained bacterial cells mixed with unstained virus-like particles (VLPs), 4) unstained bacterial cells mixed with stained VLPs. Bacterial cells for mixture 2 were stained with one microliter of dye, diluted 10X in sterile water, and incubated at room temperature in the dark for 30 min. Mixtures 1 to 3 were used as sorting controls and mixture 4 was the VT sample. All mixtures were incubated at 30 °C for 1 hour with mild rotary shaking to prevent sedimentation, and sample volumes were subsequently adjusted to 1 ml with SS before sorting using FACS. Non-specifically bound viruses were removed.

### Flow Cytometry Analyses and Sorting.

Samples were examined using an iCyt Reflection flow cytometer equipped with a 200 mW 488 nm air-cooled solid-state laser or a MoFIo™ XDP (Beckman Coulter) cytometer. Fluorescence was detected using a 520/40 band pass filter with an amplified photomultiplier tube. Events were detected using a Forward Scatter trigger and data collected in logarithmic mode then analyzed with WinList 6.0 software (Verity software house). Fluorescent polystyrene FLOW Check™ microspheres (1 μm yellow-green beads; Polysciences Inc., PA, cat #23517-10) were used as an internal standard. Samples were typically run with a concentration of 10⁵ cells ml¹.

The negative sorting controls were processed first in the following order: unstained bacterial cells, bacterial cells mixed with unstained VLPs, bacterial cells mixed with unstained viruses, and bacterial cells mixed with stained VLPs. The VT samples containing bacterial cells mixed with stained viruses were then processed, with 10,000 events usually recorded per sample to identify sorting gates which did not overlap with the negative controls. The final sample processed prior to sorting was the stained bacterial cell control to confirm that there was no overlap with the identified VT sorting gates. The flow cytometer was then cleaned prior to sorting of VT samples by repeatedly backflushing the fluidics and processing an ultra-pure water sample. For multi-cell sorts (50,000 or 100,000 cells), cells were collected in 1.5 ml low-bind tubes. For single-cell sorts, cells were sorted into 96 or 384 well plates with single-cell mode settings. The first, third and last column of the plate were left empty as negative controls, while 100 cells were collected into the second column as a positive amplification control. All sorted cells were stored at −80 °C until further processing. Flow cytometry (.fsc) files were processed for visualisation using FlowViz and FlowCore® packages.

### VT Metagenomic Sequencing.

Either VT or purified community viral samples were combined with 10 μl of DNAse I buffer and 5 μl of DNAse I (Sigma-Aldrich, #AMPD1-1KT) and incubated for 15 min at room temperature to degrade free bacterial DNA in the viral filtrate. DNAse was inactivated by adding 10 μl DNAse stop solution and incubation at 70 °C for 10 min. Viral DNA was then extracted using a low biomass DNA extraction method, which could also be applied to single cells (see below) (see, e.g., Džunková et al., loc cit. for viral DNA extraction). All reagents used in the DNA extraction were autoclaved, sterilized by 0.1 μm filtration and UV-radiated for 60 min. Each DNAsed virion sample (110 μl) was mixed with 77 μl of Lysis buffer prepared by combining 700 μl KOH stock (0.43g/10 ml), 430 μl DDT stock (Thermo Fisher, #R0861, 0.8 g/10 ml) and 370 μl water, final pH 12. The sample was incubated for 10 min at room temperature, −80 °C for one hour and 55 °C for 5 min to complete virion lysis. The alkaline reaction was then neutralized by adding 77 μl of Stop buffer (5 g Tris-HCl in 10 ml, pH 4.5) and 1 μl of Proteinase K (20 mg/ml, Sigma-Aldrich, #3115887001) was added to the sample and incubated for 30 min at 55 °C. The DNA was purified using 536 μl (1.8× sample volume) Agencourt AMPure beads (Beckman Coulter, #A63880) according to the manufacturer's instructions. Each sample was finally resuspended in 30 μl of sterile water.

Purified viral DNAs were checked for contaminating prokaryotic or eukaryotic DNA by PCR amplification of 16S rRNA genes, 18S rRNA genes and human mitochondria positive controls used were E. coli and human gDNA diluted to 0.01 ng/μl and virion-free DNA extractions used as negative controls. An optimised protocol for ultra-low input samples (cf. Rinke et al., Peer J (2016), 4: e2486) was used to prepare libraries for sequencing on the NextSeq platform. Approximately 2 GB of 2×150 bp sequence data was obtained per sample.

### Bioinformatics Analysis.

### Quality Control (QC).

Illumina data quality-control consisted of trimming ends with a quality score lower than 25 as well as sequences containing ambiguous bases, only reads longer than 100 bp were kept. Additionally, because the Illumina sequencing was done from linker-amplified DNA, it was mixed 1:1 with phiX174 DNA to minimize base-calling issues in Illumina software. Thus full-length reads matching (>98% identity) to the phiX174 genome were removed and the remaining reads were considered the target material, linkers removed and quality controlled (cf. Deng et al., Nature (2014), 513: 242-245).

### Assembly.

Contigs were assembled from post-QC reads using Velvet (Version 1.2.01) with a conservative k-mer size of 57 and the −cov cutoff option set to 10 as done previously (Hess et al., Science (2011), 331: 463-467). Iterative assembly was used whereby reads incorporated into the largest contigs were removed in to compensate for highly variable coverage (30-500X) found across the genomes in these natural samples. After 15 rounds of assembly, 26 large contigs were obtained (>30 kb that were ‘representative’ regions of the genome, see below) and referred as ‘Candidatus Genomes’. These 26 Candidatus Genomes utilized a total of ~40% of the available reads; the remaining 60% of the data presumably belong to rare members of the phage community.

### Protein Clustering.

Open reading frames (ORFs) were predicted using prodigal (cf. Richards et al., Infect Genet Evol (2011), 11: 1263-1275) from all contigs >1.5 Kb, including the CGs, as well as on all 454 reads that were not used in assembly. ORFs were clustered using CD-HIT (cf. Devirgilliis et al., Genes Nutr (2011), 6: 275-284) with a cutoff of 75% identity. Individual reads then were mapped to protein clusters using BLASTn, and evaluate a cutoff of 0.001, only non-redundant top hits were used. Rarefaction curves were calculated using a custom perl script (Rarefaction.ul). Chao-1 index was calculated from the protein cluster data as described in Chao and Lee (J A Stat Assoc (1992), 87: 210-217). Simpson diversity index (D) (see Simpson, Nature (1949), 163: 688) was calculated as D=Σn(n−1)/N(N−1). Shannon-Wiener (H′=−Σp ln p), was calculated using PHACCS (see above) (cf. Angly et al., BMC Bioinformatics (2005), 6: 41).

### Contig Annotation.

Assembled contigs >1.5 Kb were annotated as follows: ORFs were predicted using Prodigal (above) and functionally annotated using manually curated data resulting from BLASTp analyses against the non-redundant protein database of Genbank, and custom databases of T4 phage gene clusters (T4-GCs) (cf. Sullivan, Environ Microbiol (2010), 12: 3035-3056) and Microbial Metabolic Genes (cf. Sharon et al., ISME J (2011), 5: 1178-1190). To estimate the relative proportion of reads associated with particular viral types, a BLASTx search was used against the phage genomes available in NCBI, and assigned taxonomy to metagenomic reads by the taxon lineage associated with their top hit (requiring e-value <1e-3), read2family.pl available was used with the rest of the scripts.

### Hook Designing.

The inventors used the genome of the isolated Acinetobacter phages, viral contigs generated by viral tagging, and the Acinetobacter phage genomes from the NCBI RefSeq database to create a gene catalogue. The proteins of these genes were clustered into protein families (PFs) using MMseqs2, and PFs were annotated using the PHROG database. In addition, phage genomes were clustered to genus level to create viral clusters (VCs) using vConTACT2. Then, a presence-absence matrix (PAM) of PFs in VCs was created, and the most prevalent PFs and their functions were manually checked. The target PF was selected based on its prevalence in VCs and its function. For example, the inventors selected endolysin for designing the hooks for targeted isolation of phages as they were the most prevalent PF in VCs and had an important role in the bacteria lysing capacity of phages.

### Results.

### Example 1

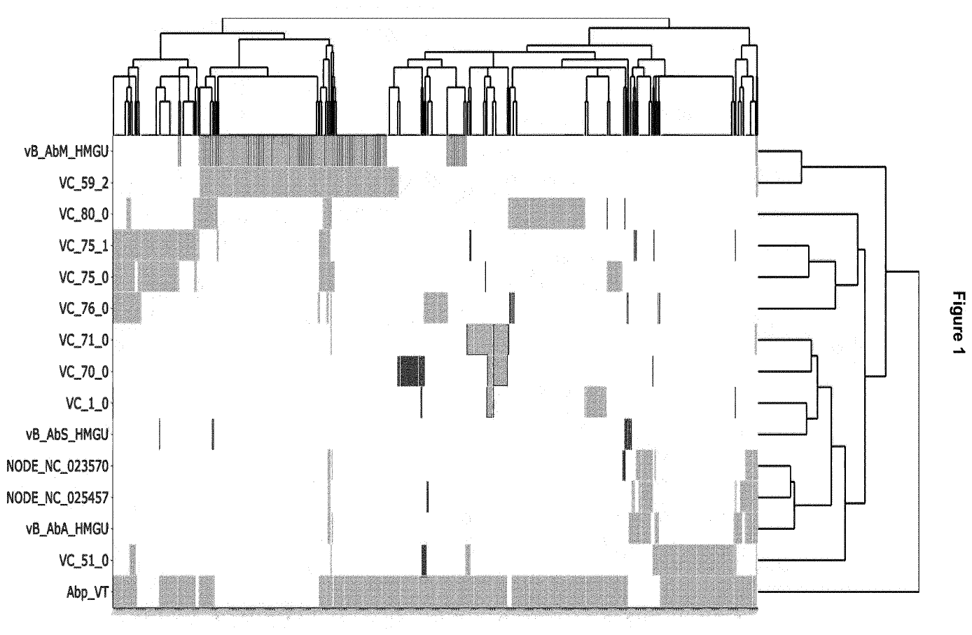

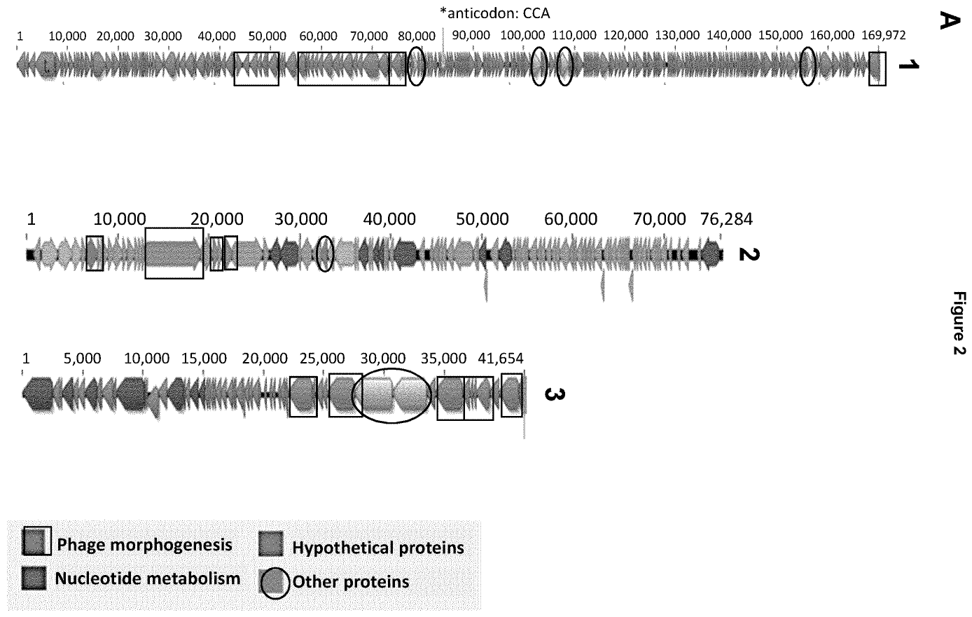

The inventors used in-depth comparative genomics and protein clustering to build a presence-absence matrix (see FIG. 1) of protein families shared between the viral clusters from the viral-tagging (Deng et al., 2014; Džunková et al., 2019) data generated against A. baumannii, also Acinetobacter phages from the NCBI RefSeq database were included. The inventors identified multiple proteins shared between these viral clusters (see FIG. 1). Protein clusters from those genes that showed the highest similarities between viral clusters were selected (see FIG. 1) as markers for developing a targeted isolation approach, which can be used to isolate novel phages from the environment and clinical samples with high antibacterial effects.

### Example 2

Using the selected proteins/genes, the inventors targeted isolated multiple phages against A. baumanii from which the inventors selected those with high efficiency against the target bacteria such as vB_AbM_HMGU (see FIG. 2A Nr. 1), vB_AbS_HMGU (see FIG. 2A Nr. 2), and vB_AbA_HMGU (see FIG. 2A Nr. 3). The inventors then further characterized their genomic and morphological features (see FIGS. 2A and B). Phage vB_AbS_HMGU showed a short latency period and high adsorption rate, making it a good candidate for phage therapy (see FIG. 2C).

### Example 3

In contrast, phage vB_AbM_HMGU and vB_AbA_HMGU showed a synergetic effect when used in combination against a multi-resistant A. baumanii strain (see FIG. 3D). Using fluorescent staining, the inventors have shown that the combination of phages vB_AbM_HMGU and vB_AbA_HMGU leads to the destruction of biofilm structure and eradicating the bacterial cells; this was not achieved by applying each phage alone (see FIG. 3). vB_AbS_HMGU shows the highest similarity to our viral-tagging data (see FIG. 1). The endolysins genes are phage-encoded peptidoglycan hydrolases used by most phages to enzymatically disrupt the bacterial cell wall at the end of their replication cycle. They have the potential to be used as independent antibacterials and, therefore, can be of interest to the pharmaceuticals industry.

### ITEMS

1. A composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in any one selected from the group consisting of SEQ ID NOs: 1-15, wherein the phages are capable of infecting Acinetobacter baumannii.

2. The composition of item 1, wherein the composition has bactericidal activity against Acinetobacter baumannii.

3. The composition of item 1 or 2, wherein the Acinetobacter baumannii bacteria are antimicrobial resistance (AMR) bacteria.

4. The composition of any one of the preceding items, further comprising a buffer.

5. The composition of any one of the preceding items, wherein the phages comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to the nucleic acid sequence encoding the amino acid sequence as depicted in any one selected from the group consisting of SEQ ID NOs: 1-15.

6. A pharmaceutical composition comprising the composition of any one of the preceding items.

7. The composition of any one of items 1-5 or the pharmaceutical composition of item 6 for use in therapy.

8. The composition of any one of items 1-5 or the pharmaceutical composition of item 6 for use in a method of treating or preventing an infection of a subject with Acinetobacter baumannii.

9. The composition or the pharmaceutical composition for the use of item 8, wherein the Acinetobacter baumannii bacteria are antimicrobial resistance (AMR) bacteria.

10. A nucleic acid, which hybridizes under stringent conditions to a signature sequence or its complementary sequence comprising a nucleic acid sequence encoding an amino acid sequence as depicted in any one selected from the group consisting of SEQ ID NOs: 1-15.

11. A nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in any one selected from the group consisting of SEQ ID NOs: 1-15, or a complementary sequence thereof.

12. The nucleic acid of item 10 or 11, wherein the nucleic acid is conjugated to a detectable label.

13. A method of preparing a composition comprising isolated phages, the method comprising:
(i) providing a sample, wherein the sample comprises phages;
(ii) targeted isolating of said phages,
   (a) which comprise a nucleic acid being part of its genome, which hybridizes under stringent conditions to a signature sequence or its complementary sequence comprising a nucleic acid sequence encoding an amino acid sequence as depicted in any one selected from the group consisting of SEQ ID NOs: 1-15, or
   (b) which comprise a nucleic acid being part of its genome, wherein the nucleic acid comprises a nucleic acid sequence, which is at least 60%, at least 70%, at least 80%, at least 90%, at least 95%, at least 98%, at least 99% or 100% identical to a nucleic acid sequence encoding an amino acid sequence as depicted in any one selected from the group consisting of SEQ ID NOs: 1-15.

14. The method of item 13, wherein the sample is any one of a naturally environmental sample, a clinical sample, a wastewater treatment plant sample, or a hospital waste resources sample.

15. A composition comprising isolated phages obtainable or obtained by the method of any one of items 13-14.

1. A composition comprising isolated phages, wherein the phages comprise a nucleic acid being part of its genome, which hybridizes under stringent conditions to a signature sequence or its complementary sequence comprising a nucleic acid sequence encoding an amino acid sequence as depicted in SEQ ID NO: 1 and wherein the composition further comprises isolated phages, wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 11,
wherein the phages comprise a nucleic acid being part of its genome, wherein the nucleic acid comprises a nucleic acid sequence, which is at least 60% identical to the nucleic acid sequence encoding the amino acid sequence as depicted in SEQ ID NO: 1 and 11
and wherein the isolated phages are capable of infecting Acinetobacter baumannii.

2. The composition of claim 1, wherein the composition has bactericidal activity against Acinetobacter baumannii.

3. The composition of claim 1 or 2, wherein the Acinetobacter baumannii bacteria are antimicrobial resistance (AMR) bacteria.

4. The composition of any one of the preceding claims, further comprising a buffer.

5. A pharmaceutical composition comprising the composition of any one of the preceding claims.

6. The composition of any one of claims 1-4 or the pharmaceutical composition of claim 5 for use in therapy.

7. The composition of any one of claims 1-4 or the pharmaceutical composition of claim 5 for use in a method of treating or preventing an infection of a subject with Acinetobacter baumannii.

8. The composition or the pharmaceutical composition for the use of claim 7, wherein the Acinetobacter baumannii bacteria are antimicrobial resistance (AMR) bacteria.

9. A kit comprising the composition of any one of claims 1-4 or the pharmaceutical composition of claim 5.

10. A kit comprising a first nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 1, or a complementary sequence thereof, and further comprising a second nucleic acid comprising a nucleic acid sequence, which is at least 60%, at least 70%, at least 80%, at least 90%, at least 95%, at least 98%, at least 99% or 100% identical to a nucleic acid sequence encoding an amino acid sequence as depicted in SEQ ID NO: 11, or a complementary sequence thereof.

11. The kit of claim 9 or 10, further comprising a detectable label.

12. A method of preparing a composition comprising isolated phages, the method comprising:
targeted isolating of phages comprised in a provided sample,
wherein the phages comprise a nucleic acid being part of its genome, which hybridizes under stringent conditions to a signature sequence or its complementary sequence hereto comprising a nucleic acid sequence encoding an amino acid sequence as depicted in SEQ ID NO: 1 and further comprise a nucleic acid being part of its genome, which hybridizes under stringent conditions to a signature sequence or its complementary sequence hereto comprising a nucleic acid sequence encoding an amino acid sequence as depicted in SEQ ID NO: 11,
and wherein the phages comprise a nucleic acid being part of its genome, wherein the nucleic acid comprises a nucleic acid sequence, which is at least 60% identical to the nucleic acid sequence encoding the amino acid sequence as depicted in SEQ ID NO: 1 and 11.

13. The method of claim 12, wherein the sample is any one of a naturally environmental sample, a clinical sample, a wastewater treatment plant sample, or a hospital waste resources sample.

14. A composition comprising isolated phages obtainable or obtained by the method of any one of claims 12-13.
